(19) United States
(12) Patent Application Publication
(10) Pub. No.: US 2025/0248658 A1
(43) Pub. Date: Aug. 7, 2025

(54) A DEVICE AND METHOD FOR THE DETECTION AND MONITORING OF CARDIOVASCULAR DISEASE

(71) Applicant: 3 AIM IP PTY LTD, North Sydney, New South Wales (AU)

(72) Inventors: Gaetano GARGIULO, Kogarah, New South Wales (AU); Neil Lawrence ANDERSON, North Sydney, New South Wales (AU); Ritaban DUTTA, Battery Point, Tasmania (AU)

(21) Appl. No.: 18/854,726

(22) PCT Filed: Apr. 5, 2023
(86) PCT No.: PCT/AU2023/050273

§ 371 Date: Oct. 7, 2024

(30) Foreign Application Priority Data
Apr. 8, 2022 (AU) 2022900926

(51) Int. Cl. A61B 5/00; A61B 5/021; A61B 5/318; A61B 7/00; G16H 40/60

(52) U.S. Cl. CPC A61B 5/7246; A61B 5/02116; A61B 5/318; A61B 5/4842; A61B 5/746; A61B 7/00; G16H 40/60; A61B 2562/06

(57) ABSTRACT

A device for heart monitoring, the device comprising a processor having a digital signal processing unit, wherein the digital signal processing unit is configured to receive and process physiological signals from at least one sensor assembly having one or more sensors. The processor comprises a program having executable instructions that when executed on the processor, the processor is configured to execute the steps of: synchronising processed signals for each sensor assembly; mapping the synchronised signals as waveforms for each sensor; predetermining waveform amplitudes and predetermined time intervals to calculate at least one cardiac function parameter as a data value; and performing a step of differential analysis between the calculated data value and a set of referencing cardiac health parameters to determine a condition of the heart.

### TECHNICAL FIELD

The present invention relates to a device and a method for heart monitoring. The device and a method for heart monitoring may relate to a human heart or to an animal heart. More particularly, a method for calculation of various parameters related to heart function, in particular to the early detection of heart failure. The method may use at least one sensor assembly, and more preferably in combination with an electrocardiogram electrode placed in one or more standard auscultation positions.

### BACKGROUND

Heart failure (HF) is a condition that develops when a person's heart doesn't pump enough blood for their body's needs. This can happen if their heart can't fill up with enough blood. It can also happen when the heart is too weak to pump properly. The term “heart failure” does not mean that the heart has stopped. However, heart failure is a serious condition that needs medical care.

There is an estimated 26 million people with heart failure (HF) worldwide. The prevalence of known HF is 1.5-2% of the adult population. Heart failure can develop suddenly (the acute kind) or over time as a person's heart gets weaker (the chronic kind). It can affect one or both sides of the heart. Left-sided and right sided heart failure may have different causes. Most often, heart failure is caused by another medical condition that damages the heart. This includes coronary heart disease, heart inflammation, high blood pressure, cardiomyopathy, or an irregular heartbeat.

Heart failure may not cause symptoms right away. But eventually, you may feel tired and short of breath and notice fluid build-up in the lower body, around the stomach, or neck. Heart failure can also damage the liver or kidneys. Other complications include pulmonary hypertension or other heart conditions, such as an irregular heartbeat, heart valve disease, and sudden cardiac arrest. Doctors usually classify patients' heart failure according to the severity of their symptoms. The list below describes the most commonly used classification system, the New York Heart Association (NYHA) Functional Classification. It places patients in one of four categories based on how much they are limited during physical activity.

The Class Patient Symptoms: I: No limitation of physical activity. Ordinary physical activity does not cause undue fatigue, palpitation, dyspnea (shortness of breath); II: Slight limitation of physical activity. Comfortable at rest. Ordinary physical activity results in fatigue, palpitation, dyspnea (shortness of breath); III: Marked limitation of physical activity. Comfortable at rest. Less than ordinary activity causes fatigue, palpitation, or dyspnea; IV: Unable to carry on any physical activity without discomfort. Symptoms of heart failure at rest. If any physical activity is undertaken, discomfort increases.

The Class Objective Assessment: A: No objective evidence of cardiovascular disease. No symptoms and no limitation in ordinary physical activity; B: Objective evidence of minimal cardiovascular disease. Mild symptoms and slight limitation during ordinary activity. Comfortable at rest; C: Objective evidence of moderately severe cardiovascular disease. Marked limitation in activity due to symptoms, even during less-than-ordinary activity. Comfortable only at rest; D: Objective evidence of severe cardiovascular disease. Severe limitations. Experiences symptoms even while at rest.

For example, a patient with minimal or no symptoms but a large pressure gradient across the aortic valve or severe obstruction of the left main coronary artery is classified: Function Capacity I, Objective Assessment D. And a patient with severe anginal syndrome but angiographically normal coronary arteries is classified: Functional Capacity IV, Objective Assessment A.

Currently, heart failure is a serious condition that has no cure. Typically, patients are symptomatic before treatment which is often too late to have a significant effect on quality of life and cost of care. There is a clear need to detect HF earlier to enable early interventions to prevent or at least delay, the onset of the disease. Treatment such as healthy lifestyle changes, medicines, some devices and procedures can help many people have a higher quality of life, especially if implemented early. It is important to note, the later the treatment (ie the higher the NYHA classification) the more expensive the treatment is for both length of hospital stay, amount of care required and the cost of the treatment eg heart pacemaker, a left ventricular assist device or even a heart transplant.

Patients with more advanced disease can be monitored in a clinical setting with a pressure catheter in either the left or right chamber of the heart although the use of Echocardiography is an essential investigation in patients with suspected heart failure. An echocardiogram—a form of ultrasound; provides assessment of cardiac chamber size and structure, ventricular function, valvular function and key haemodynamic parameters. Echocardiogram may be performed before and after exercise with the latter studying heart function under stress at a higher heart rate.

In addition, the jugular venous pulse exam typically using ultrasound is an important aspect of assessing a patient's volume status, especially in patients with heart failure, liver failure and kidney failure. Elevated jugular venous pressure is a manifestation of abnormal right heart dynamics, mostly commonly reflecting elevated pulmonary capillary wedge pressure from left heart failure. This usually implies fluid overload, indicating the need for diuresis.

Another parameter called heart rate variability (HRV) using ECG signals is a fundamental and non-invasive techniques used to assess autonomic modulation of the heart. Traditional HRV has been shown to be markedly reduced in patients with HF and this reduction has been related to the severity of HF and its prognosis.

The gold standard for continuous remote monitoring is an implantable arterial sensor that transmits blood pressure and heart rate. The cost of such devices and procedures may be in order of USD$25,000 with the risk of adverse events.

There is long felt need for an externally applied device that can measure key biometrics related to cardiac function that are indications of changes in mechanical function of the heart for patients that have been identified as being at risk of developing heart failure.

Any discussion of the prior art throughout the specification should in no way be considered as an admission that such prior art is widely known or forms part of common general knowledge in the field.

### SUMMARY

### Problems to be Solved

It is an advantage to provide an externally applied device that can measure key biometrics related to cardiac function that are indications of changes in mechanical function of the heart for patients that have been identified as being at risk of developing heart failure.

It is also an advantage to use the device and system in a clinical setting at a face-to-face consultation as well as in a virtual care/home setting.

It is also an advantage to allow for analysing the data in real time via a telehealth consultation.

It is also an advantage to provide the device that would be able to give similar information to larger pieces of capital equipment such as ultrasound (including an echocardiogram) but is a portable, easy to use and low cost.

It is an advantage to derive information from the device that would be key aspects within the cardiac cycle such as cardiac conduction times (when used with an ECG), heart rate variability, valve actions, ejection time, refill times, contractility, heart elasticity, vessel compliance, hemodynamic function and ejection fraction, as well as changes in the jugular venous pulse that are representative of the pressures in the right atrium.

It is an advantage to provide an easier assessment of cardiac function without the use of large equipment that needs to be used by experts. This would allow more regular measurements to be taken as an indication of disease progression and the effect of treatment.

It is also an advantage to provide a device or a sensor that measures key early signs of heart failure with some relevant biometrics including but not limited to changes in cardiac contraction force or displacement, and central blood pressure and pulse transit times from apex to the aortic arch or the suprasternal notch.

It is also an advantage to combine the device of the sensor together with the software of determining an early warning score for heart failure based on the received signals from the sensor or sensor dot.

It is also an advantage to provide a method to estimate the ejection time and refill time from the sensor as well as to obtain a central and peripheral hemodynamic picture of the human body.

It is an object of the present invention to overcome or ameliorate at least one of the disadvantages of the prior art, or to provide a useful alternative.

### Means for Solving the Problem

A first aspect of the present invention may relate to a device for heart monitoring, the device comprising: a processor having a digital signal processing unit, wherein the digital signal processing unit is configured to receive and process physiological signals from at least one sensor assembly having one or more sensors; the processor comprises a program having executable instructions that when executed on the processor, the processor is configured to execute the steps of: synchronising processed signals for each sensor assembly; mapping the synchronised signals as waveforms for each sensor; eliminating signals with artefacts from the analysis process; predetermining waveform amplitudes and predetermined time intervals to calculate at least one cardiac function parameter as a data value; or combining a number of cardiac function parameters into an algorithm and performing a step of differential analysis between the calculated data value and a set of referencing cardiac health parameters to determine a condition of the heart.

Preferably, the sensor assembly comprise a force sensitive resistor, a displacement sensor, and a pair of electrocardiogram electrodes; wherein the pair of electrocardiogram electrodes are embedded in or attached using wires to the sensor assembly. Preferably, the program further comprising non-transition memory configured to allow the processor to: storing a condition data value corresponding to a first use of the at least one sensor assembly at a predetermined position; and comparing a second condition data value corresponding to a second use of the least one sensor assembly at the predetermined position, the processor is configured to determine the progression of heart condition based on the difference in condition data values from the first use and the second use. Preferably, the processor is further configured to: providing an alert to the subject, when the determined progression is worse compared to the previous use.

Preferably the first use may be before exercise and the second use may be after exercise. Preferably the first use is a point in time that is used to compare the values from a later second use of the system. The second use may be to assess the progress of the disease over time, which may be days, or weeks, or months; in which the assessment may include values of specific treatment where treatment could be medication, medical device use, supplements, diet changes and/or exercise. The second use may also be to be soon after the first use (for example within minutes from first use) which may be following an exercise regime that is designed to put the cardiovascular system under stress which may be from use on an exercise bike or treadmill. The compared values may be used a good predictive tool for the presence or progression of cardiovascular disease which may not be limited to for example, coronary artery disease. As the amplitudes will be higher post exercise, the higher amplitudes are not considered artefacts and is important to analyse the increase from rest and post exercise and monitor how long it will take for the amplitude to return to the normal sized amplitude. One non-limiting example of normalisation of data may be to take a calculated time interval and divide by the calculated length of a cardiac parameter with another cardiac parameter. An example may be taking two cardiac function parameters such as a Rwave to another Rwave of a cardiac cycle in which the time interval is between the time of the Rwave and the time of the another Rwave. It may be appreciated that the interval can be a Pwave to another Pwave or that the interval can be a particular cardiac function parameter to a different cardiac function parameter in a cardiac cycle.

Another aspect of the present invention may relate to a device for heart monitoring, the device comprising: a processor. A first sensor assembly comprising a first group of sensors, the sensor assembly positioned at a predetermined location of a subject, wherein the first group of sensors is configured to receive physiological signals. The first group of sensors comprises a pair of electrocardiogram electrodes in communication with a digital signal processing unit of the first sensor assembly for communicating the first group of physiological signals to the processor. The processor comprises a program having executable instructions that when executed on the processor, the processor is configured to: process the first group of physiological signals at predetermined intervals to obtain representations as data values; and determine at least one heart function or an indication of heart health based on analysis of processed data values.

Preferably, the first group of sensors comprise a force sensitive resistor and a displacement sensor.

Preferably, the processor is adapted to measure heart rate variability, contractility and cardiac conduction times based on physiological signals received from the force sensitive resistor, the displacement sensor, and the electrocardiogram electrode, when the sensor assembly is positioned over or in the vicinity of a heart of the subject. An alternative is when the processor is adapted to measure the pulse characteristics from the right atrium when the sensor assembly is positioned over the vicinity of the jugular vein on the lower neck of the patient.

Preferably, the processor is configured to determine events from a cardiac cycle based on received physiological signals, wherein the determined events correspond to at least one heart function selected from the group of: closure of the semilunar valves, ventricular blood refill period, cardiac conduction time, cardiac contractility, ejection period, cardiac output valves opening period, cardiac output valves closing period and when placed over the jugular vein, the change in pressures in the right atrium.

Preferably, the device further comprises a second sensor assembly comprising a second group of sensors, wherein the second sensor assembly is positioned at a different predetermined location to the first sensor assembly.

Preferably, the first sensor assembly is positioned over the heart, and the second sensor assembly is positioned on a peripheral location such as the arm, neck, or leg the processor is configured to derive the duration of first and second cardiac sounds. More preferably, the processor is configured to derived the duration by the low frequency components of the signals at which the sensor assemblies detect.

Preferably, the processor is configured to derive the pulse transit time.

Preferably, the processor is configured to derive central blood pressure and vessel stiffness based on relative timing between aortic valves opening and closing and waveform shapes of the received physiological signals. More preferably, the processor is configured to derive central blood pressure by using two sensors on the chest. For example, a sensor may be disposed on a first region on the chest, and another sensor may be disposed on a second region on the chest. Another example for deriving central blood pressure may use a sensor disposed on a region on the chest and another sensor disposed on the neck. Another example for deriving central blood pressure may use a sensor disposed on the chest and another sensor disposed on the iliac region.

Preferably, the processor is configured to derive the difference in amplitudes of the force sensors at predetermined time intervals in the force signals to determine data values for: A) calibrated peak amplitude for expansion, and B) calibrated peak amplitude for contraction, in which only a single force sensor is disposed on a first region on chest or in which the first force sensor is disposed on a first region on the chest and the second force sensor is disposed on a second region on the chest.

Preferably, the processor is configured to derive elasticity of the vessel based on a ratio of A:B.

Preferably, the processor is further configured to derive the timing difference of the received signals from displacement sensors relative to the electrocardiogram electrode to determine data values for: C) time at which the heart and/or vessel expands, and D) time at which the heart and/or vessel contracts.

Preferably, the processor is configured to generate an indication of the central hemodynamic function of the subject from a ratio of (A/C):(B/D) obtained from different predetermined locations of the sensor assemblies. More preferably, the first and second sensors are disposed on a first location and a second location on the chest respectively.

Preferably, the processor is configured to synchronise physiological signals received from the first sensor assembly and the second sensor assembly at two different locations.

Preferably, the processor is configured to determine an ejection fraction of the heart based on subtracting physiological signals from the displacement sensor in the first sensor assembly and the second sensor assembly.

Preferably, the first sensor assembly is positioned over the cardiac apex, and the second sensor assembly is positioned over the suprasternal notch, the processor is configured to determine at least one heart function through the differential analysis step.

Preferably, the first sensor assembly is positioned over the cardiac apex of the subject, and the second sensor assembly is positioned at the aortic auscultation position of the subject, the processor is configured to determine the at least one heart function relating to the left chambers of the heart through the differential analysis step.

Preferably, the first sensor assembly is positioned over the cardiac apex of the subject, and the second sensor assembly is positioned at the pulmonary valve auscultation position of the subject, the processor is configured to determine the at least one heart function relating to the right chambers of the heart through the differential analysis step.

Preferably, the first sensor assembly and the second sensor assembly are positioned at adjacent locations over the top of the heart, the processor is configured to derive pulse elasticity and pulse timing, which then the processor determines arteria stiffness and blood pressure through the differential analysis step.

Preferably, the differential analysis step comprising the step of determining the status of the jugular venous pulse, based on physiological signals received from a first sensor assembly which correlates to various pressure changes in the right atrium, when the sensor assembly is positioned over or in the vicinity of the jugular vein in the neck of the subject.

Preferably, wherein the processor further comprises the step of: annotating specific morphological features, including potential artefacts after the step of mapping the synchronised waveforms for each sensor.

Preferably, the processor further comprises the steps of: calculating the average and measurement of variance for each of the amplitudes and time intervals, removing identified artefacts; joining selected signal parts to form a cleaned signal including cardiac cycle, recalculating the new average and measurement of variances after the step of predetermining waveform amplitudes and predetermined time intervals.

Preferably, the processor further comprises the steps of: calculating the average and measurement of variance for each of the amplitudes and normalised time intervals, removing identified artefacts; joining selected signal parts to form a cleaned signal including cardiac cycle, recalculating the new average and measurement of variances after the step of predetermining waveform amplitudes and predetermined time intervals.

Preferably, the calculated time intervals, for the at least one cardiac function parameter, are normalised with respect to length of cardiac cycle.

Preferably, the device further comprises non-transition memory configured to store received physiological signals from the sensor assemblies forming historical data.

Preferably, the processor is configured to monitor heart condition over time based on comparing derived data values from latest measurement, and historical data values, when the sensor assemblies were positioned at the same predetermined location.

Preferably, the program comprises a set of predetermined parameters with data value thresholds for categories of heart function and heart health, wherein the processor is configured to compare the analysed data values to the set of parameters, which allows the processor to identify the heart function and indication of heart health when the analysed data value is within the data value threshold.

Another aspect of the present invention may relate to a method for the measurement of specific heart functions, the method comprising the steps of: a. using at least one sensor assembly where each sensor assembly is made up of one or both of a force sensor and a displacement sensor; b. combining the one or more sensor assemblies together with at least one pair of electrocardiogram electrodes which is separate to or included in the sensor assembly; c. placing the sensor assembly and electrocardiogram electrodes on the outside of the chest over, or adjacent to, the heart at specific locations depending on what functions are to be measured; d. recording the resulting signals and processing these signals to determine a value or range of values which equate to specific heart function parameter or parameters.

Preferably, the processor calculates the relative timing of specific signal features and the heart functions are contractility, cardiac conduction times, valve actions, ejection time and refill time. Preferably, the relative timing between aortic valves opening and closing and their waveform shape is correlated to central blood pressure and vessel stiffness. Preferably, the ratio of calibrated ejection time/refill time correlates to the ejection fraction normally measured with cardiac ultrasound or other imaging devices. Preferably, the force sensor and piezo sensor are calibrated and one of the sensor assemblies is placed over the apex of the heart and another one adjacent to the top of the heart. Preferably, the processor calculates the difference in amplitudes of the force sensors at specific points in the force signals which correspond to calibrated peak amplitude (A) for expansion and one for contraction (B). Preferably, the ratio of specific differences in amplitude (A/B) are used to calculate elasticity of the vessel. Preferably, the processor calculates the timing difference of specific locations of the signals from the piezo sensors relative to the electrocardiogram and these differences correspond to vessel expansion receiving the high-pressure pulse (C) and vessel contraction spreading out the pulse (D). Preferably, the ratio of the calculated peak amplitude (A) divided by the timing (C) for expansion and the calculated peak amplitude (B) divided by the timing (D) of contraction elasticity (A/C)/(B/D) is an indication of vessel compliance. Preferably, the processor calculates and compares the ratio (A/C)/(B/D) at different locations giving an indication of the central hemodynamic function of the patient.

Preferably the processor calculates the heart rate and the resulting heart rate variability at each sensor location. Preferably, the processor subtracts the signals from the two calibrated piezo sensors and then calculates the differences in amplitude at specific points of the subtracted signals in the piezo signals where the difference in amplitude correlates with the ejection fraction. Preferably, one sensor assembly is located at the cardiac apex and one sensor assembly is located at the suprasternal notch position resulting in a calculation of overall heart function. Preferably, the overall heart function is at least one selected from the group of: cardiac contractility, central blood pressure, ejection fraction, timing of valves opening and closing, blood refill time, and blood ejection time. Preferably, one sensor assembly is located at the cardiac apex and one sensor assembly is located at the aortic auscultation position resulting in the calculation of heart functions related to the left chambers of the heart. Preferably, one sensor assembly is located at the cardiac apex and one sensor assembly is located to the pulmonary valve auscultation position resulting in the calculation of heart functions related to the right chambers of the heart. Preferably, the two sites are the adjacent to the top of the heart and any other targeted arterial pulse locations (e.g. iliac crest, radial etc . . . ) and the measurement of pulse elasticity as well as pulse timing are taken to establish peripheral arteria stiffness and blood pressure. Preferably the sensor is located over the jugular vein to measure jugular venous pressure. Preferably, where together the calculations form the different sites give an indication of overall hemodynamic performance. Preferably, measurements are simultaneously taken at more than two pulse sites. Preferably, measurements are used to monitor the effect of pressure altering medication. Preferably, the resulting values, or change in values over time, of the calculated heart function parameters are used to detect heart disease or the advancement of heart disease. Preferably, the method is performed using manual annotation using appropriate software. Preferably, the method is performed automatically using dedicated algorithms. Preferably, any artefacts are removed using manual processing. Preferably, any artefacts are removed using automatic processing. Preferably the algorithms that calculate specific parameters have built in methodology to allow for removed artefacts from any of the signals. Preferably, the detection of heart disease or the advancement of heart disease is performed in either a clinical or virtual setting.

Another aspect of the present invention may relate to a device for heart monitoring, the device may comprise a processor having a digital signal processing unit, which may preferably be assisted with machine learning based AI processing unit, wherein the digital signal processing unit may be configured to receive and process physiological signals from at least one sensor assembly having one or more sensors, in which the sensors are in communication with or in conjunction with the AI processing unit to learn and preprocess the signals to remove any unwanted artefacts in the data; the processor may comprise a program having executable instructions that when executed on the processor, the processor is configured to execute the steps of:
synchronising processed signals for each sensor assembly;
mapping the synchronised signals as waveforms for each sensor;
conducting an AI based smart template matching to score similarity of all available cardiac cycles, and to find ideal parts from the signal to identify signal parts related to desired cardiac cycles;
annotating specific morphological features, including potential artefacts; automatically join all selected signal parts to form a cleaned signal including all desired cardiac cycles;
predetermining waveform amplitudes and predetermined time intervals to calculate at least one cardiac function parameter as a data value; calculating the average and measurement of variance for each of the amplitudes and time intervals, removing identified artefacts; replacing with typical values;
recalculating the new average and measurement of variances and then performing a step of differential analysis between the calculated data value and a set of referencing cardiac health parameters to determine a condition of the heart.

Preferably, the heart may be a human heart or an animal heart.

Preferably, the differential analysis may take into account four main measures of variability such as range, interquartile range, measurement of variance, and variance; so any one of them could be used. The range may be the difference between the highest and lowest values; the interquartile range may be the range of the middle half of a distribution; the measurement of variance may be the average distance from the mean; the variance may be the average of squared distances from the mean.

In the context of the present invention, the words “comprise”, “comprising” and the like are to be construed in their inclusive, as opposed to their exclusive, sense, that is in the sense of “including, but not limited to”.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1 is a diagram of typical cardiac ultrasound procedure where 101 is the ultrasound probe, 102 is the conical ultrasound beam, and 103 is the cross section of the heart.

FIG. 2 shows a typical image (201), identifying the blood path (202), the aortic valve (203), and the atrioventricular valve (204).

FIGS. 3A to 3F shows the timing of the ultrasound relative to an electrocardiogram (ECG). And FIGS. 3A to 3F shows images of the ECG relative to various stages of the heart's mechanical status.

More particularly, FIG. 3A shows an image of the ECG relative to the P-wave onset, where all valves are closed and atria are refilling;

FIG. 3B shows an image of the ECG relative to the P-wave body, where atria are contracting and valves to ventricles open;

FIG. 3C shows an image of the ECG relative to the P-wave offset, where atria completed mechanical action and valves close;

FIG. 3D shows an image of the ECG relative to the QRS onset, where ventricles filled with blood start depolarizating. The relative pressure differences between the atria-ventricles ensure valves are sealed (first deflection peak on dots notes the conduction time);

FIG. 3E shows an image of the ECG relative to the QRS body offset, where ventricle contraction (contractility noted by large waveform and peak on dots);

FIG. 3F shows an image of the ECG relative to the QRS-offset/T-wave onset, where Aorta valve open and blood shifts to aorta.

FIG. 4 shows the signals from over a subject's heart using the device or Sensor dot. Signal 409 is from the ECG, Signal 410 is from the piezo sensor, and Signal 411 is from the force sensitive resistor (FSR) sensor.

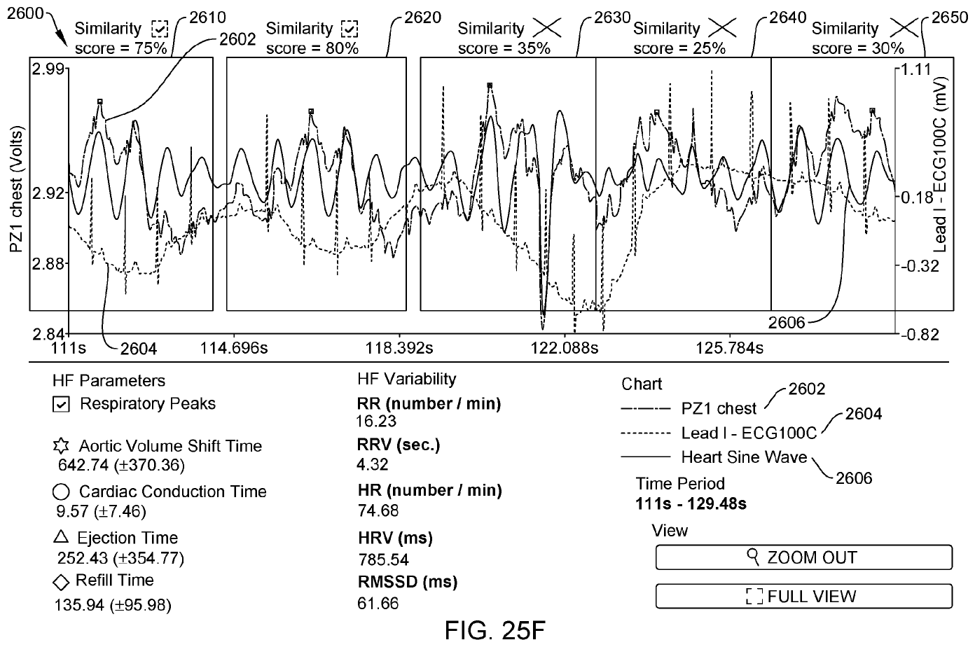

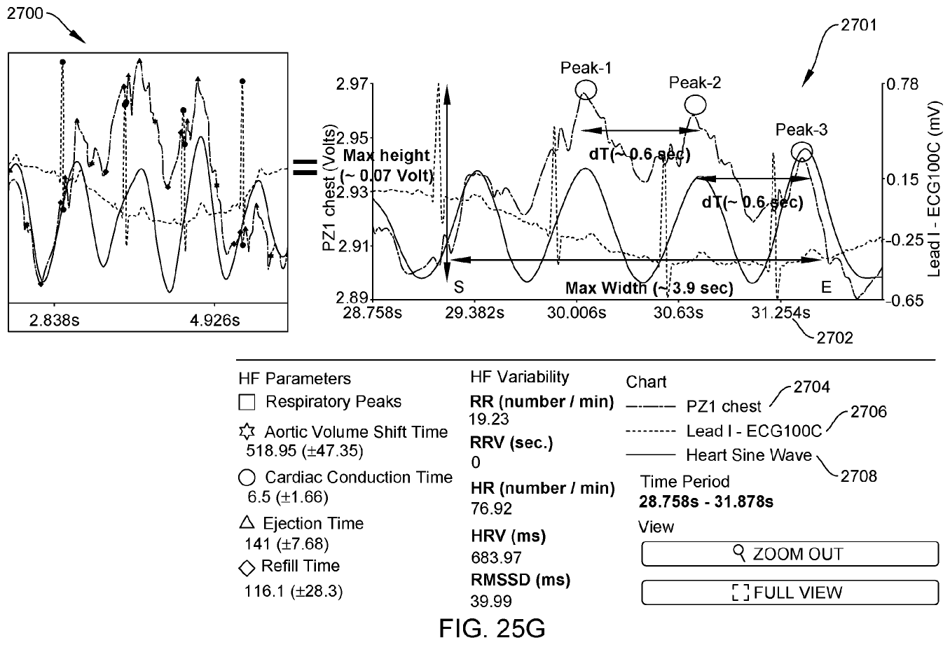

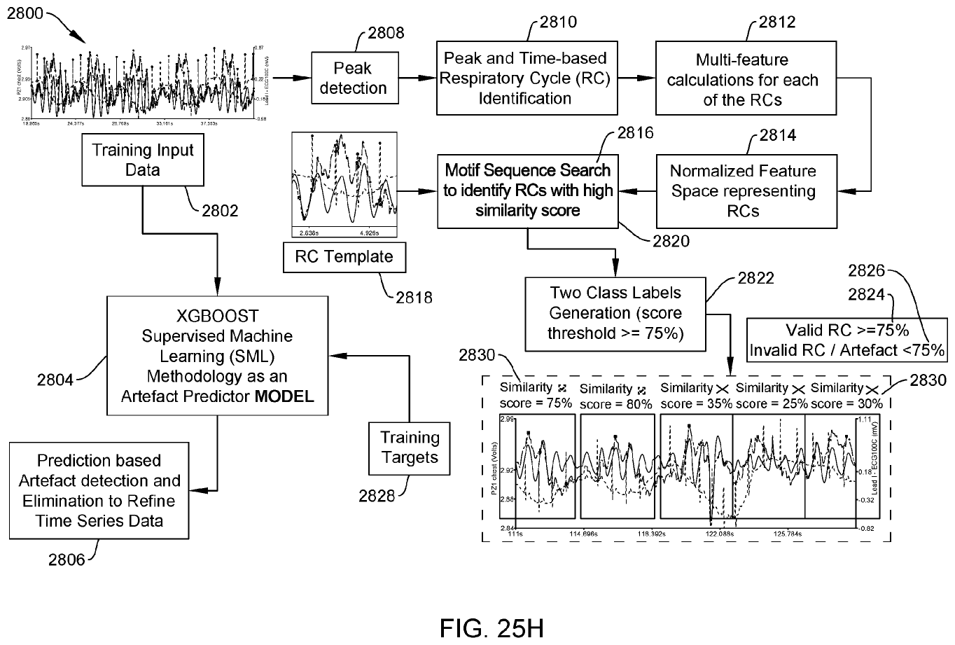

In FIG. 4, 401 to 408 shows the quantitative timing from various pairs of different morphological features on the signals through a cardiac cycle. More particularly, 401 shows the atrial contraction period from the piezo signal which in the Cardiac Cycle corresponds to closure of the semilunar (aortic and pulmonary) valves while the mitral and tricuspid valves are open. 402 shows the peak of the R wave of the ECG to a trough in the piezo signal which corresponds to the cardiac conduction time. 403 shows the ventricular isovolumetric contraction period (with all valves closed) with the ventricles contracting due to the QRS signal from the ECG. Cardiac contraction which is known as the tension developed and velocity of shortening. That is, the ‘strength’ of contraction of myocardial fibers at a given preload and afterload 404 is the ejection period from when the aortic and pulmonary valve open to when they close (405), showing rapid ejection leading to relaxation due to the ECG t wave. It is a measure of the time it takes to eject the blood from the heart. 406 corresponds to the indicative time of when the tricuspid and mitral valves open leading to blood starting to flow back into the ventricles. The time between 405 to 407 is called the Isovolumetric Relaxation time which is the heart relaxing with all valves closed. 408a corresponds to when there is rapid filling into the ventricles leading to a period of slower filling (408b). For ease of understanding Location 1 is identified in two cardiac cycles showing the corresponding refilling periods in each cycle. The rapid filling and the slower passive filling of the ventricles in general results in 95% of the ventricles being full with the remaining 5% being pushed in during atrial contraction (401). The cardiac cycle then repeats itself.

FIG. 5 shows the relationship between ECG, the sensor dot (on the chest and arm) with the chest signal differentiated to get the actual acoustic sound signal. The blood pressure pulse is shown at the bottom from the Biopac gold standard device.

The cardiac sound signal shows the region corresponding to S1 corresponds to the mitral and tricuspid valves closing and corresponds to the pulse. Note the delay of the pulse as it reaches the finger (the bottom Biopac signal) and is indicative of 503 Pulse Transit Time (PTT). The second heart sound (S2) represents closure of the semilunar (aortic and pulmonary) valves.

FIG. 6 shows two sensor dots positioned at the apex 601 and suprasternal notch 602, which correspond to the proximity of two key auscultation locations at the 5^{th} intercostal space and above the 2^{nd} intercostal space.

FIG. 7 shows the timing on the sensor dot to plot the central hemodynamic profile. At which the period (1) corresponds to vessel expansion receiving the high-pressure pulse while the period (2) corresponds to vessel contraction—spreading out of the pulse. (A) corresponds to the calibrated peak amplitude for expansion and (B) corresponds to the calibrated peak amplitude for contraction.

FIG. 8 illustrates the amplitudes of interest as indicated at 702-A and 702-B from the piezoelectric waveform when the displacement sensors of the first sensor assembly and the second sensor assembly is positioned over the apex of the heart and over the suprasternal notch respectively. And showing the calculation of the ejection fraction=B as % of A.

FIG. 9 is a table showing how the program may calibrate certain parameters and timing for the list of cardiac functions. The heart rate variability (HRV) can be detected from ECG and/or from a force sensitive resistor (FSR) or piezo sensor.

FIG. 10A shows a table showing sensor ID/type to be downloaded when particular signals are downloaded/displayed.

FIG. 10B shows the sensor types corresponding to the sensor ID of FIG. 10A.

FIG. 10C is the legend of the sensors in FIG. 10B.

FIG. 11A shows the various sensor positions over the chest of the patient.

FIG. 11B shows the various sensor positions over the finger or wrist of the patient.

FIG. 11C shows the various sensor positions over the neck or carotid of the patient.

FIG. 11D shows the various sensor positions over the pelvic region of the patient.

FIG. 11E shows the various sensor positions over the femoral/popliteal/posterior tibial regions of the patient.

FIG. 12 shows the synchronisation of the ECG, chest piezo and the chest FSR waveforms. The processor may have magnified signal and performed a time calibration to produce the resulting graph.

FIG. 13 shows the magnified signal as waveform and amplitude calibration of the FSR cardiac apex and the FSR suprasternal notch positions.

FIG. 14 shows how the program may allow the processor to graph parameters with respect to time/date measured. In this particular example, the left ventricular ejection time is used as an example when the patient is undergoing treatment. It may be appreciated that other parameters may also be graphed with respect to date measured.

FIG. 15 shows an example updatable care plan based on results from physiological measurements from the sensor assemblies.

FIG. 16 shows which predetermined time intervals and amplitudes to measure from for determining the pulse transit time (PTT). In this particular example, the PTT from the cardiac apex to suprasternal notch is measured. The processor may be configured to derive or estimate the central pressure based on these waveforms. It may be appreciated that a similar processing may determine the PTT between two other locations.

FIG. 17 shows how the processor may estimate blood pressure based on calibrating the waveforms generated, in which peak to peak pressure may be obtained with a single point calibration and can be compared with beat by beat non invasive blood pressure.

FIG. 18 shows how the processor may determine different tissue compliance. In this example, the chest waveforms (top 4807) based on signals received from a front FSR and a back FSR from a sensor assembly vs the wrist waveforms (bottom 4808) based on signals received by a front FSR and a back FSR from another sensor assembly.

FIG. 19 shows a schematic diagram of the morphic band/sensor which may receive physiological signals from an area of the body, eg chest area. The signal is transmitted to a processor with a digital processing unit, in which the program may be an artificial intelligence software which when the parameters as outlined in Table 2 is processed, the processor may determine the risk or potential for heart failure (as shown as green/amber/red in the representative monitor in the figure. The program may then generate a care plan which may recommend medication(s), exercise and/or diet to actively and proactively start managing to reduce the risk as much as possible so as to working towards avoiding heart failure. The process may be scheduled at predetermined date intervals so that the system and program can monitor progression and/or monitor any worsening of cardiac function or cardiac parts. And quickly adapt the care plan in view of current measurements. This Figure also shows a clinical flow diagram for monitoring a patient with heart failure

FIG. 20 displays the objective of the overall system for early heart failure detection.

FIG. 21A shows the timing of the jugular venous pulse (JVP) displayed in relation to the carotid arterial tracing, first (S1) and second (S2) heart sounds, and the electrocardiogram (ECG).

FIG. 21B shows the simultaneous carotid and venous pulse tracings taken with a sensitive form of Lombard's tambour. x-x′, relative position of writing points with arcs. This figure illustrates the method of marking c-waves as well as the different terminologies, indicative carotid artery and jugular venous pulse waveforms

FIG. 21C (top) illustrates four curves showing transition of right auricular pulse to supraclavicular venous pulse. I—pressure changes in right auricle; II—pressure changes in intrathoracic veins; III—volume changes of neck vein; IV—supraclavicular pulse taken with tambour. FIG. 21C (bottom) illustrates the supraclavicular venous pulse (upper) and subclavian pulse (lower).

FIG. 22 shows a typical jugular venous pulse from an ultrasound probe before and after exercise.

FIG. 23 shows the jugular venous pulse (JVP) when recorded from both a sensor band and a sensor dot.

FIG. 24 shows the signals from a neck band when the subject is at various positions in a tilt table test emphasising that signals change as blood pressure changes in tilted positions illustrated.

FIG. 25A shows an image of a screen of a computer based analytical software system, which displays sensory signals (for example, Piezo DOT and ECG), that are automatically annotated for various morphological features that correspond to key cardiac parameters. Analytical software system automatically detects and removes the artefacts in the sensory signals before analytically annotate the remaining parts of the signal to estimate measurement of variance of the key cardiac parameters.

FIG. 25B shows an image of a screen of a computer based analytical software system, which displays the artefacts in the sensory signals [marked in an oval with a ‘X’], that are required to be removed to make the signals more realistic before the automatic annotation. The good segments of the sensory signals are marked in an oval with a ‘√’. The system automatically eliminates the artefacts in the signal before estimating measurement of variances. Removal of artefacts thereby reduces the measurement of variances of cardiac parametric calculations and thereby advantageously reduces the time at the cardiac parameters are calculated.

FIG. 25C shows an image of a screen of a computer base analytical software system, which displays the effects of artefacts on the cardiac parametric calculation in the real-life sensory signals. In the presence of signal noise and artefacts, measurement of variance of the key cardiac parameters, namely, Aortic Volume Shift, cardiac conduction time, ejection time etc. are too high and erroneous. The system automatically eliminates the artefacts part of the sensory signal before estimating measurement of variances. Removal of artefacts reduces the measurement of variances of cardiac parametric calculations which also advantageously reduces the time at the cardiac parameters are calculated.

FIG. 25D shows an image of a screen of a computer base analytical software system, which displays the effects of artefacts on the cardiac parametric calculation in the real-life sensory signals, after automatically removing significant artefacts. The system automatically eliminates the artefacts part of the signal before estimating measurement of variances. The removal of artefacts reduces the measurement of variances of cardiac parametric calculations. The measurement of variance of the key cardiac parameters, namely, Aortic Volume Shift, cardiac conduction time, ejection time etc. are within clinically accepted ranges, hence the sensory data and the associated analytics are proven to be accepted by the clinician.

FIG. 25E shows an image of a screen of a computer base analytical software system, which displays the effects of artefacts on the cardiac parametric calculation in the real-life sensory signals, after automatically removing significant artefacts. Rapid template matching using machine learning is advantageous to eliminate artefacts automatically. This example figure showcase a selected template as a gold standard respiratory cycle based on piezo sensor. This template is used to score each of the recorded respiratory cycles based on similarity. The higher the similarity score represents a better match, hence indication of a good cycle, in which the data can be accepted clinically. A lower similarity score on the other hand indicates potential artefacts or other kinds of noisy signal and cycle.

FIG. 25F shows an image of a screen of a computer based analytical software system, which displays an example of the cardiac parametric calculation in the real-life sensory signals, which depicts the methodology of the automatically rejecting artefacts cycles within the recorded time series signal. Matching analysis based on machine learning or a machine learning engine involves an advanced motif template matching, using angular positions of multi-feature points, within the time period defined by a respiratory cycle. This automatic visual approach mimics a cognitive interactive method to match and eliminate artefacts. Similar score is a predictive outcome of a trained learning algorithm, automatically identifying artefacts cycles to be removed to clean the time series data.

FIG. 25G shows an image of a screen of a computer based analytical software system focusing on the region as outline in the template 2598 for a piezo based respiratory cycle (as indicated in FIG. 25E), which is also shown in the box 2620 for a piezo based respiratory cycle (as indicated in similarity score=80% in FIG. 25F). FIG. 25G shows how the automated machine learning algorithm to match and find all valid respiratory cycles that exist in the recorded time series data using predefined thresholds to identify an ideal match of a valid respiratory cycle recorded using the piezo sensor.

FIG. 25H shows a schematic diagram of the artefact elimination methodology in the real-life sensory signals obtained by the sensors, depicting the methodology of the automatically rejecting artefacts cycles within the recorded time series signal.

### DESCRIPTION OF THE INVENTION

Preferred embodiments of the invention will now be described with reference to the accompanying drawings and non-limiting examples. Embodiments of the present disclosure relate to sensing systems and methods for monitoring heart functions and/or provide an indication of heart health based on processed physiological signals received from one or more sensor assembly.

In a preferred embodiment of the invention, a device 1000 may comprise at least one sensor assembly 1002, where the sensor assembly 1002 may have a group of sensors associated each other. The group of sensors may use a force sensor 1004 such as a force sensitive resistor (FSR), and a displacement sensor 1006 such as a piezoelectric sensor. Using both sensors will simultaneously measure force displacement of the subject as well as the velocity of such displacement (from the piezoelectric sensor 1006). The compression force and the dynamic force that may be exerted on the force sensor 1004 may be used to calibrate or adjust the displacement velocity signal generated by the displacement sensor 1006. This calibration may allow for accurate and continuous direct measurement of speed or velocity of displacement of the skin as well as the displacement itself. As such, accurate and continuous measurement of blood impulse and therefore cardiac impulse can be obtained from movement of the skin alone.

It may also be an advantage to combine the use of an electrocardiogram (ECG) electrode 1008 or lead 1008 in the group of sensors. The ECG lead may be embedded in the sensor assembly. The electrocardiogram electrode 1008 may receive signals of how often the heart beats or the heart rate (leading to the calculation of heart variability), as well as how regularly it beats or the heart rhythm. The information from the signals of the ECG is that it can provide the clinician important information of the patient's heart, for instance about the contractility of the heart, possible narrowing of the coronary arteries, or an irregular heartbeat. The signals simultaneously generated by the group of sensors synergistically and advantageously provide more and accurate identification or estimate of blood ejection time/period as well as blood refill time/period of the ventricles.

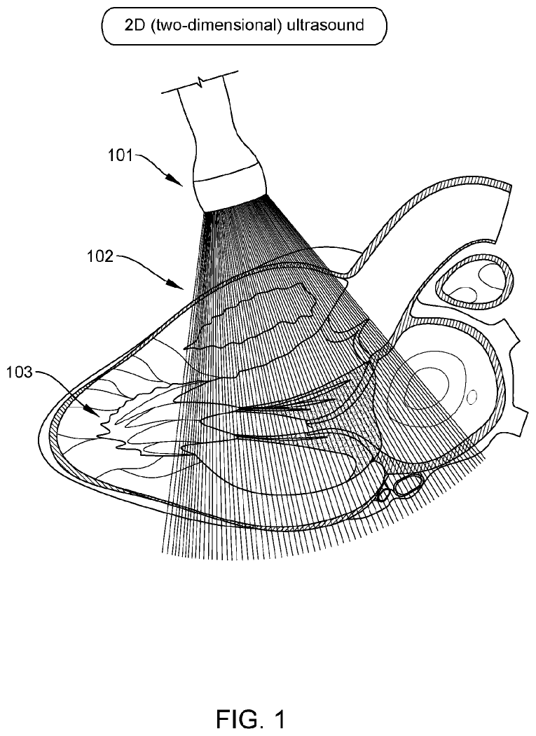

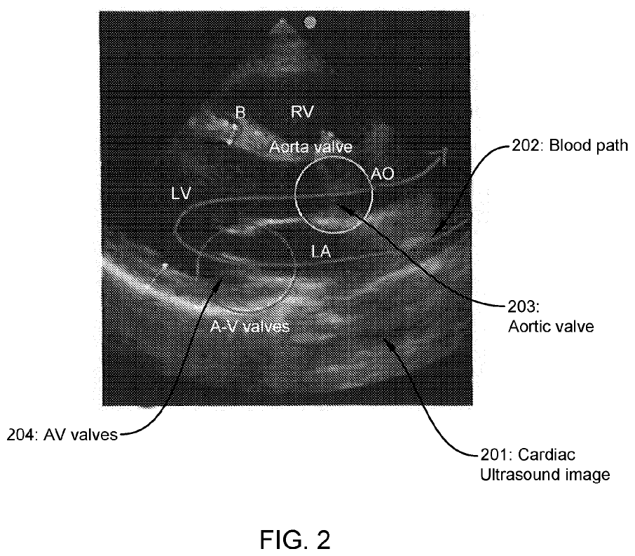

As shown in FIG. 1, 2D (two dimensional) ultrasound could be used to verify the timing obtained by the sensor assembly 1002. FIG. 1 shows a diagram of a typical cardiac ultrasound procedure where the ultrasound probe or transducer 101 is used for emitting a conical ultrasound beam 102 or ultrasound pulses 102 into the tissue of interest, which in this case is the heart of the subject. The image of the cross section of the heart 103 is depicted as representative of the tissue of interest. When the ultrasound pulses 102 encounter the tissue, a portion of the pulse or waves is reflected back to the transducer 101 and the fraction of the returning waves depends on the density and size of the tissue examined. This ultrasound procedure is used in clinical settings and is called an echocardiogram, wherein it uses sound waves to produce images of the heart. This procedure or common clinical test allows a doctor to see the heart beating as well as the functioning of the heart such as in the process of pumping blood. The images from an echocardiogram are used to identify heart disease. FIG. 2 shows a representative cardiac ultrasound image 201, which can identify the blood path 202 as well as other heart parts such as the heart valves for example, the aortic valve 203 and the atrioventricular valve 204. While ultrasound can be used, ultrasound machines are expensive and there is a long felt need to create a low-cost sensor assembly that the person can easily couple onto their skin to obtain the data as using ultrasound as well as obtaining further physiological signals which can be analysed and used to determine the heart function as well as heart health of the subject. The procedure of ultrasound can be a means of verification in a clinical setting to the sensor assembly used.

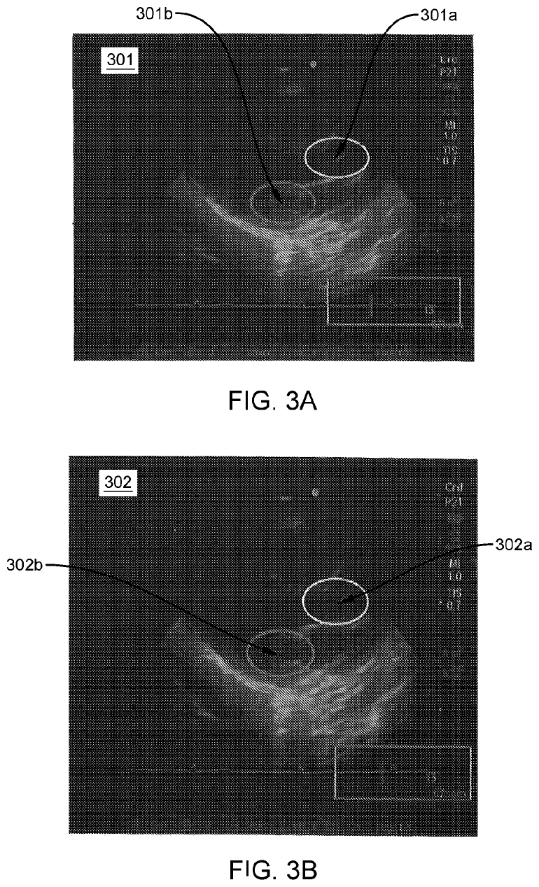

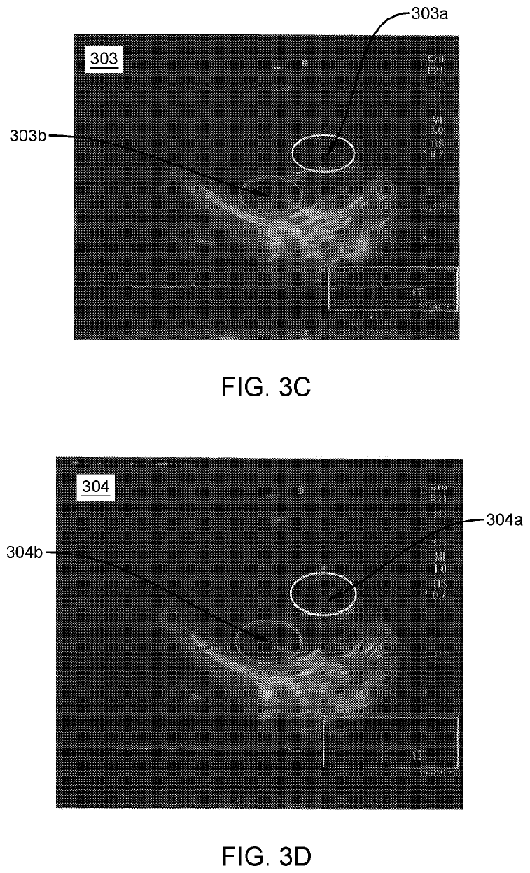

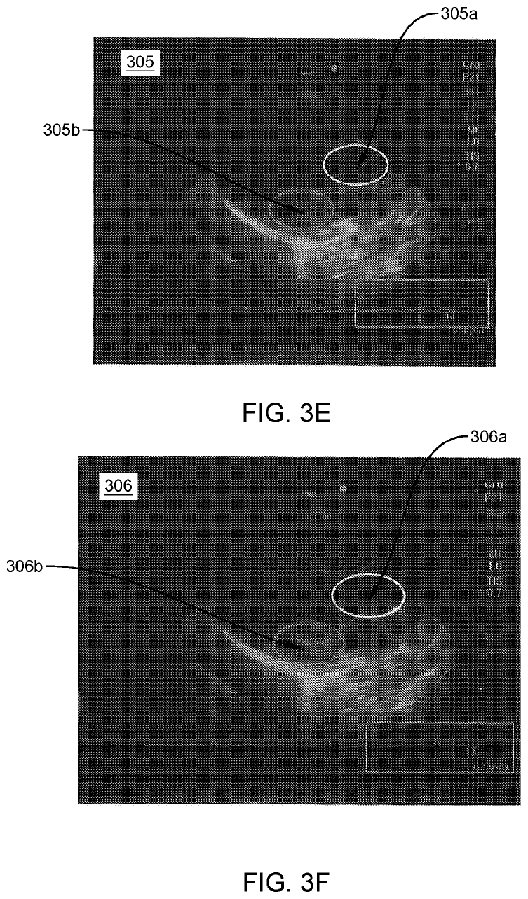

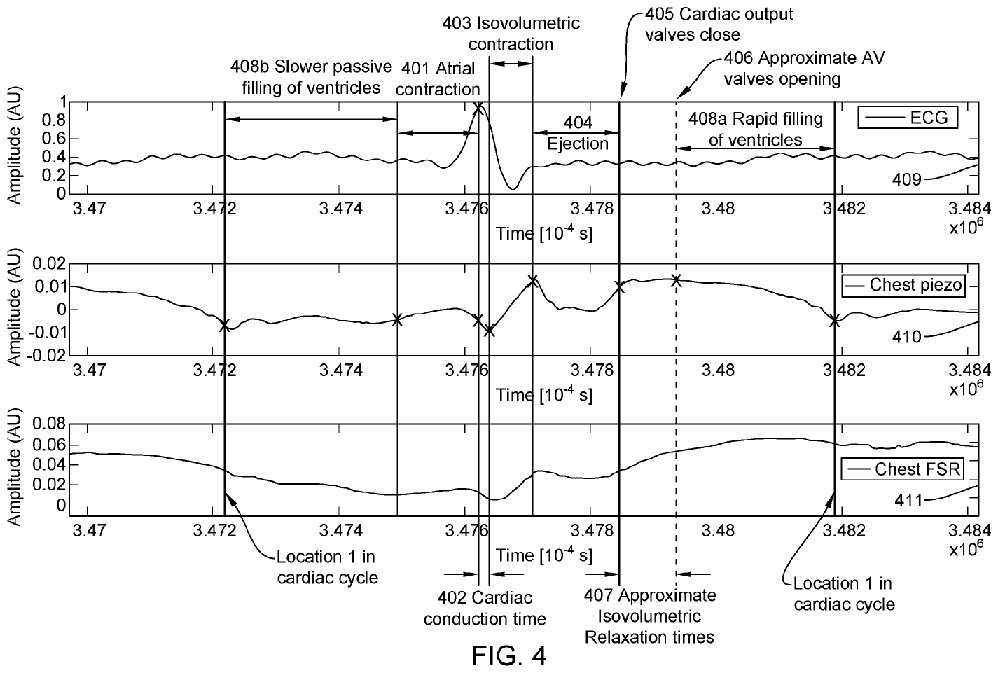

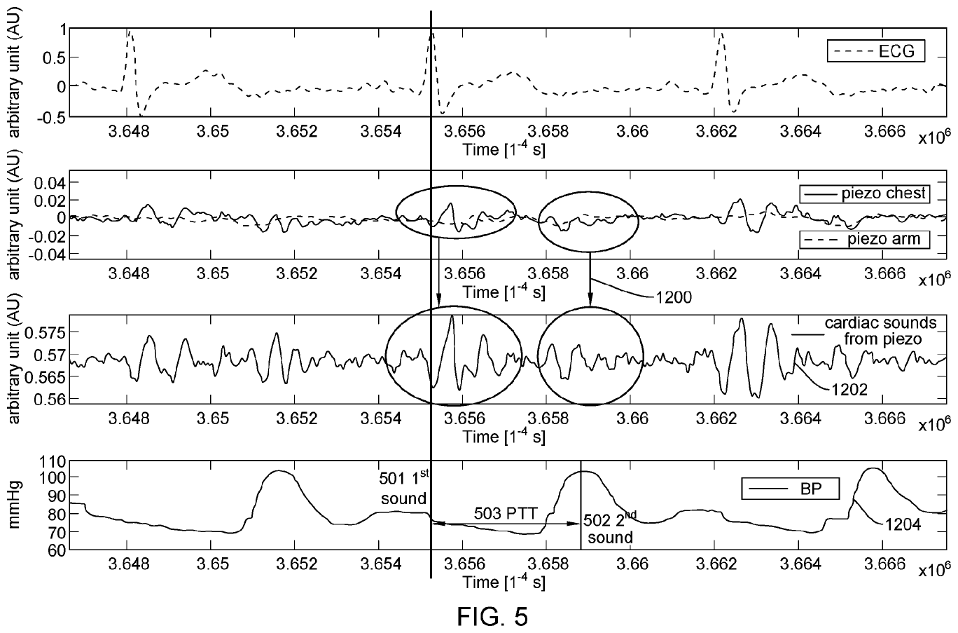

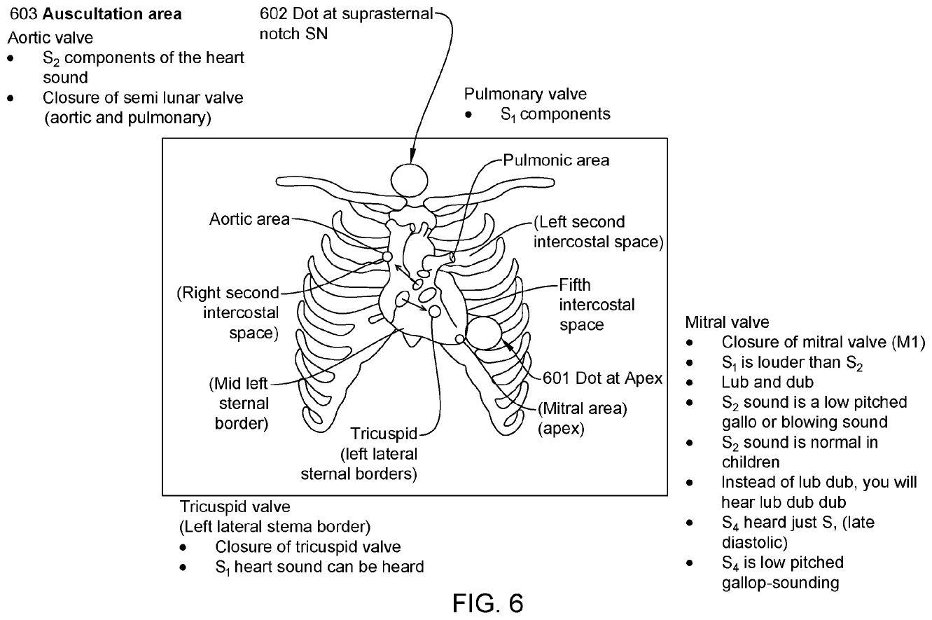

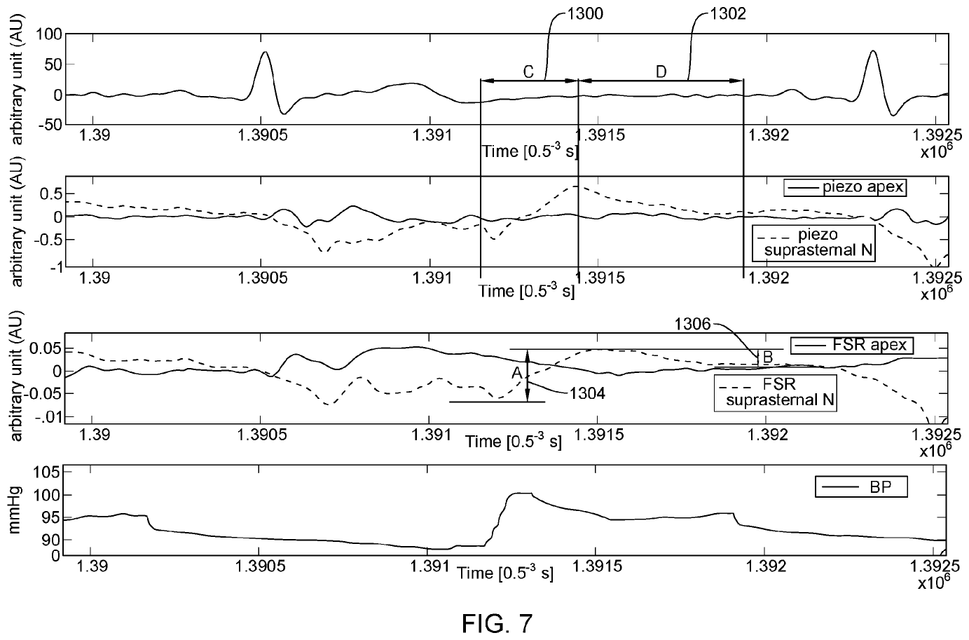

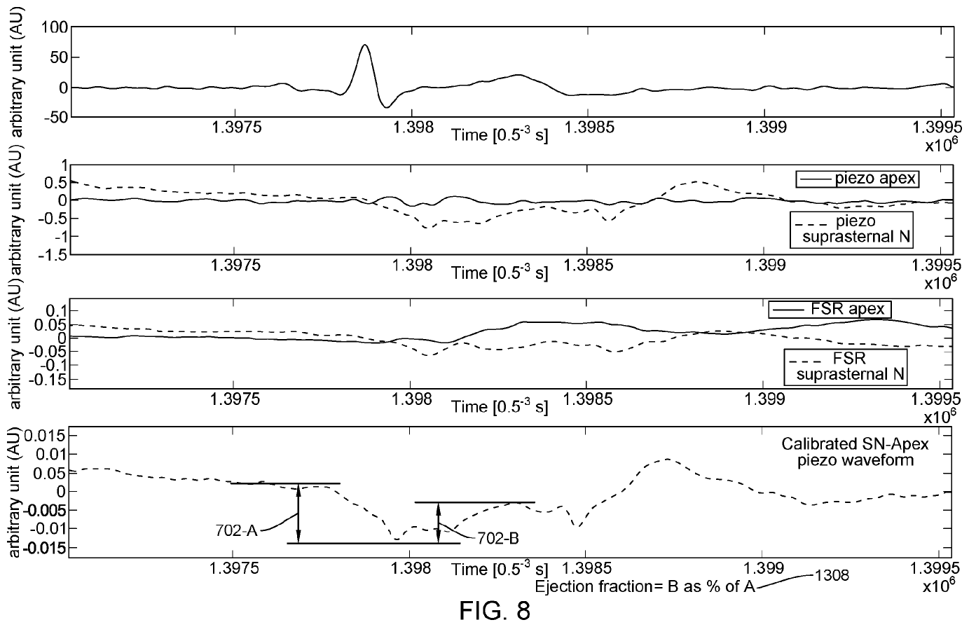

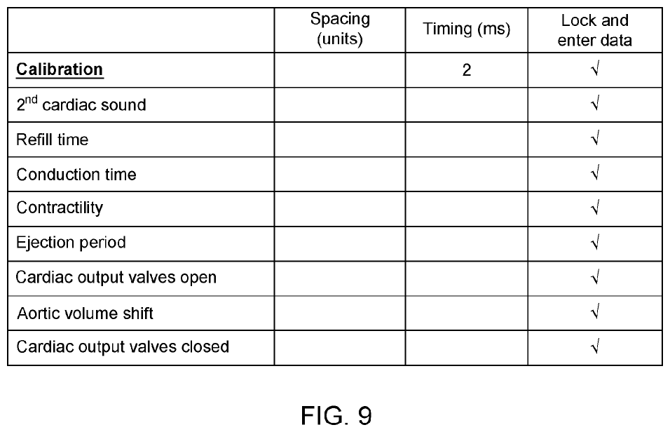

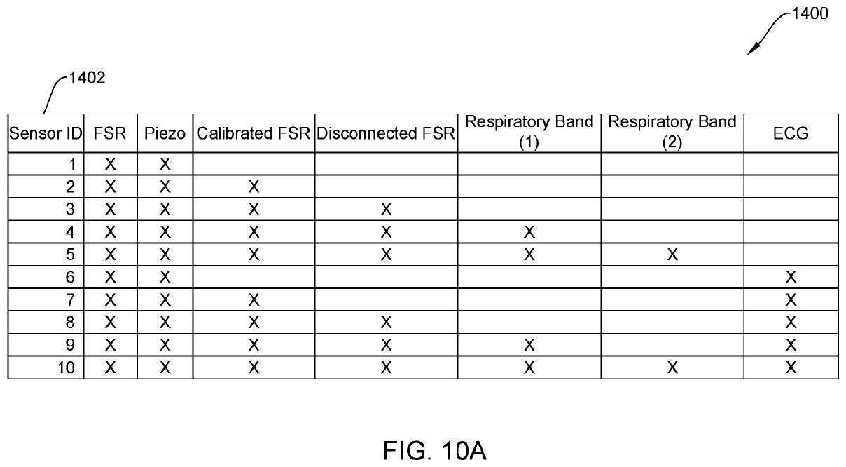

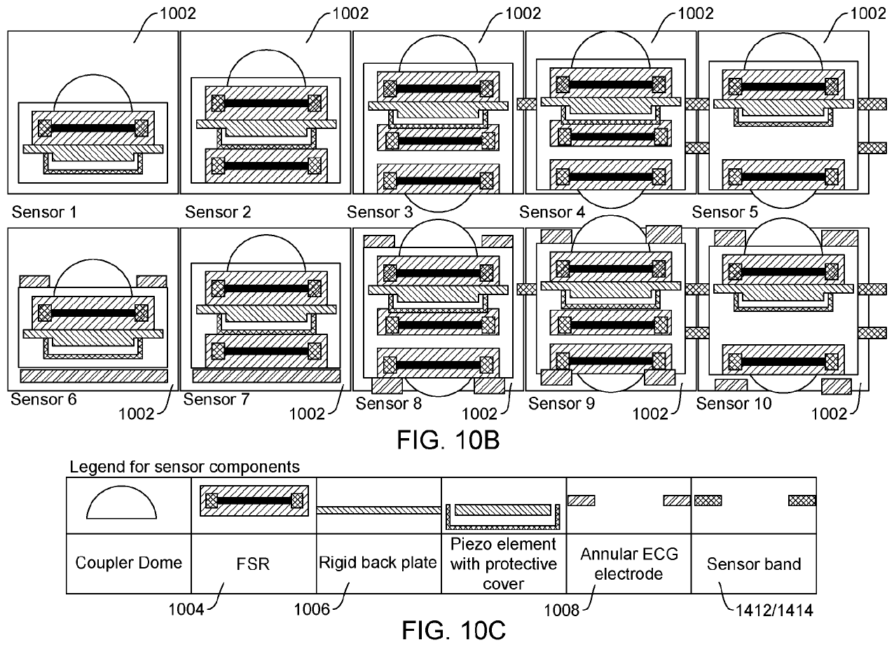

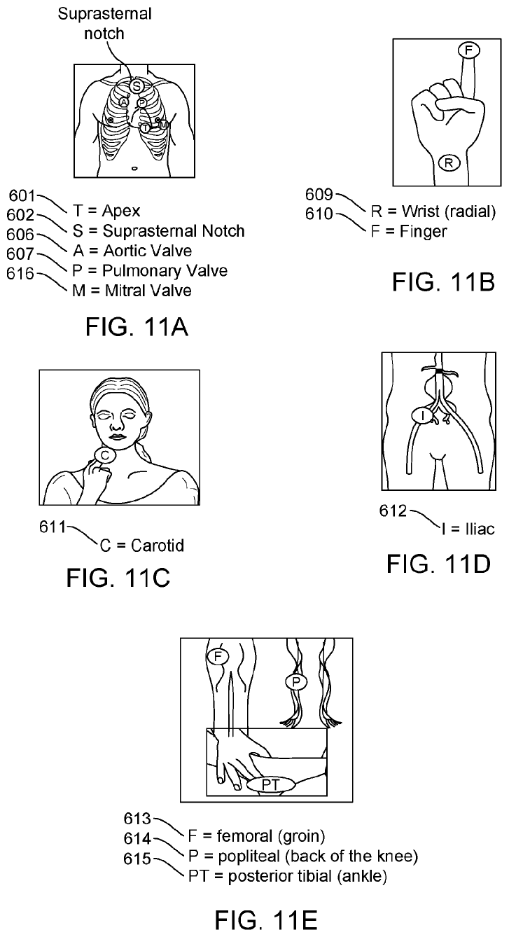

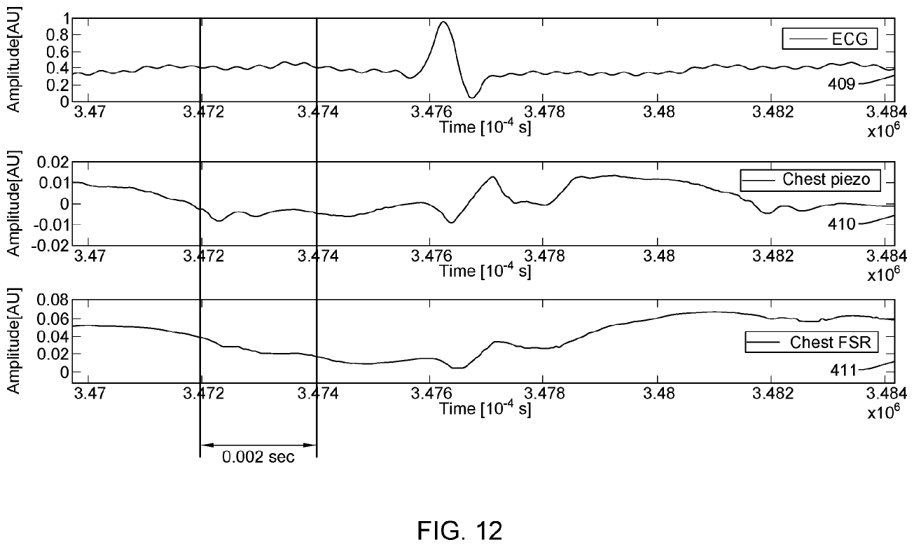

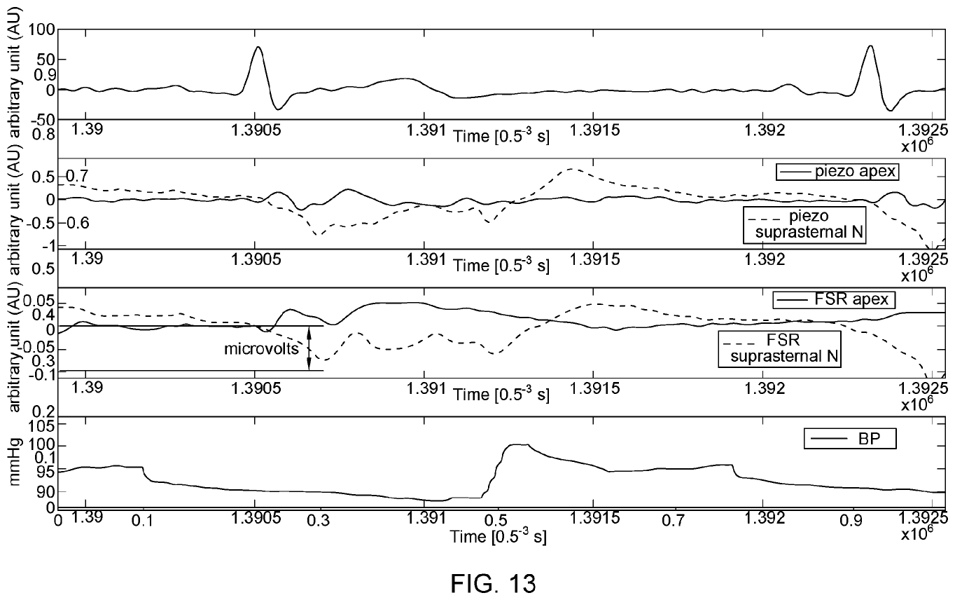

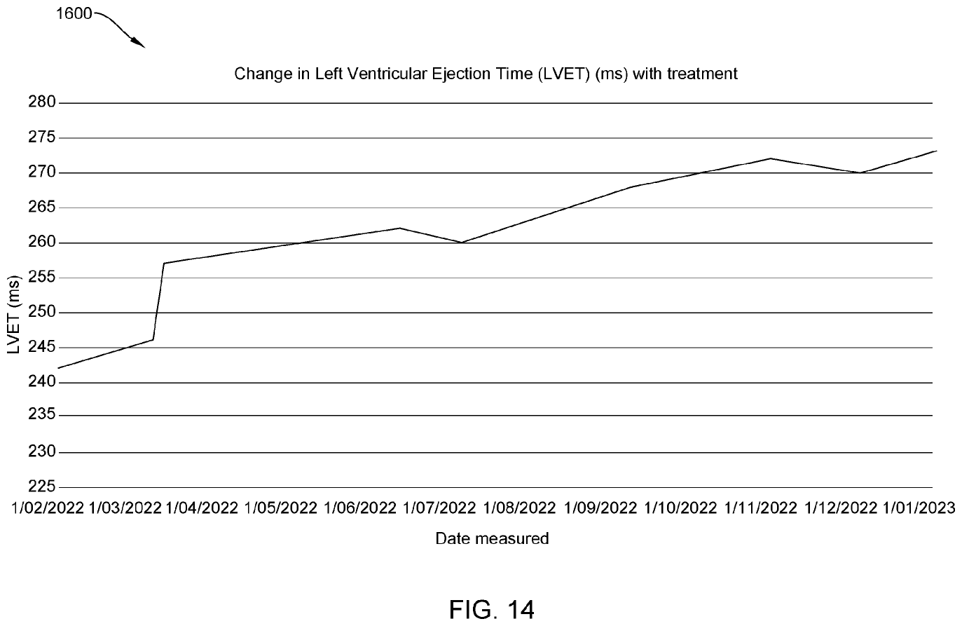

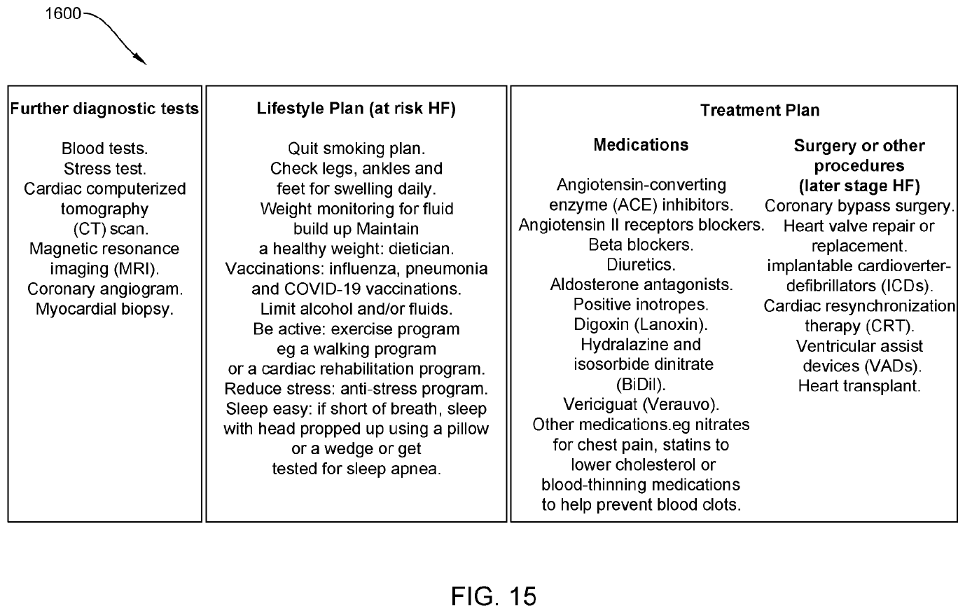

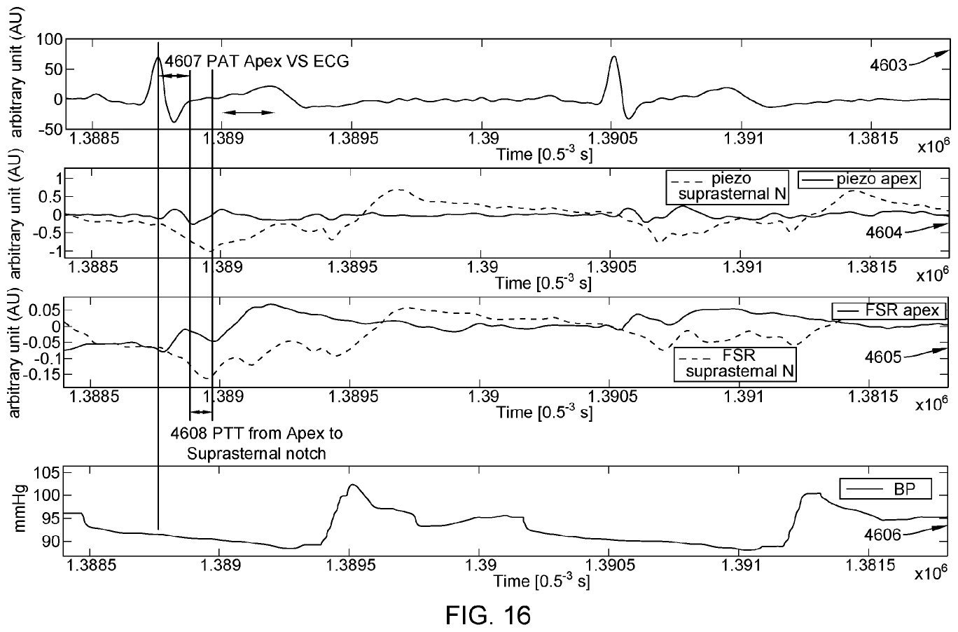

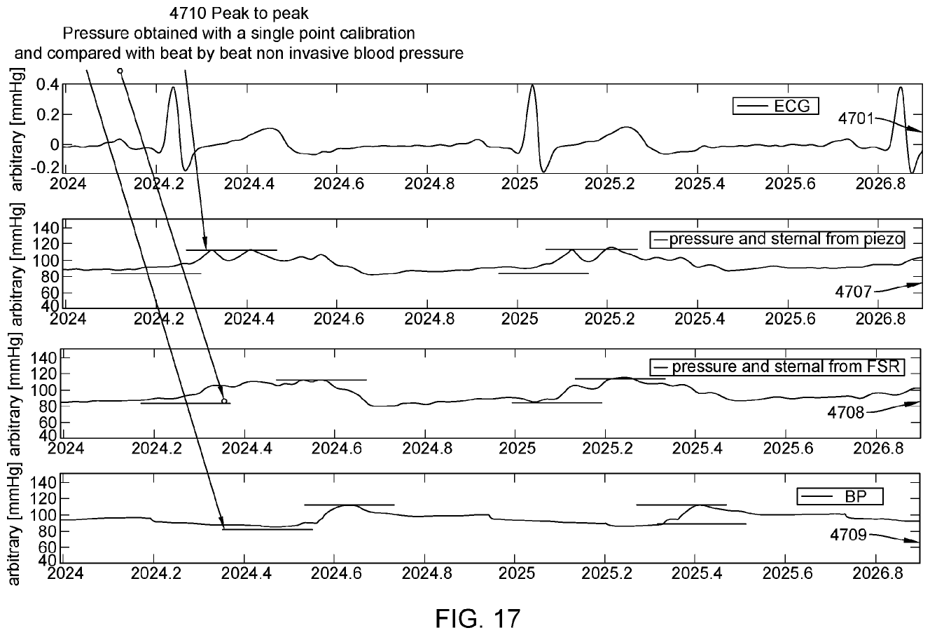

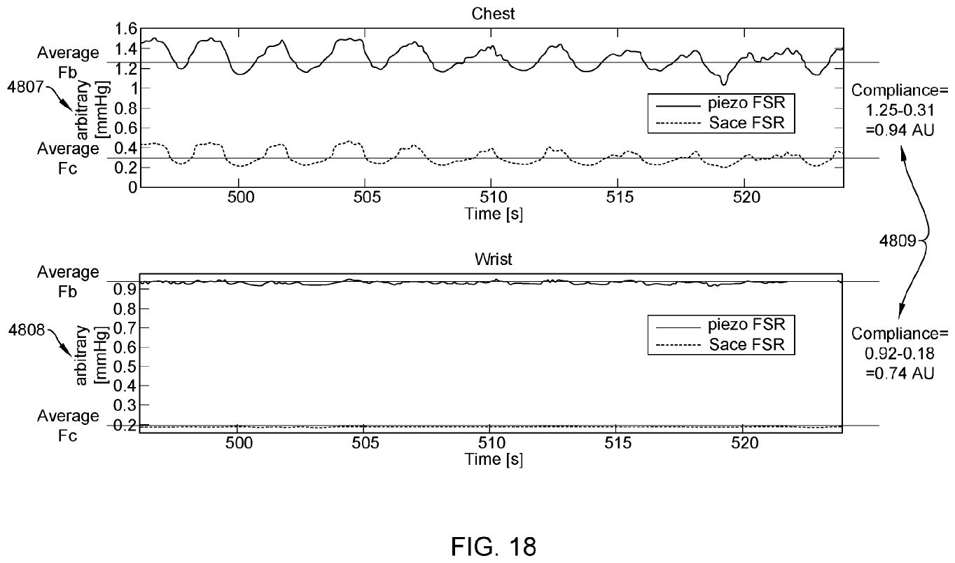

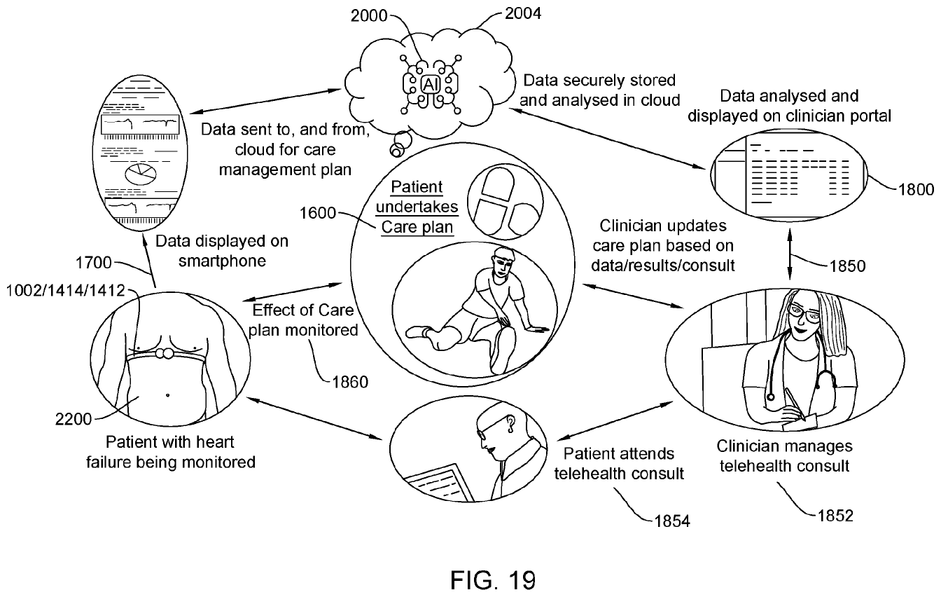

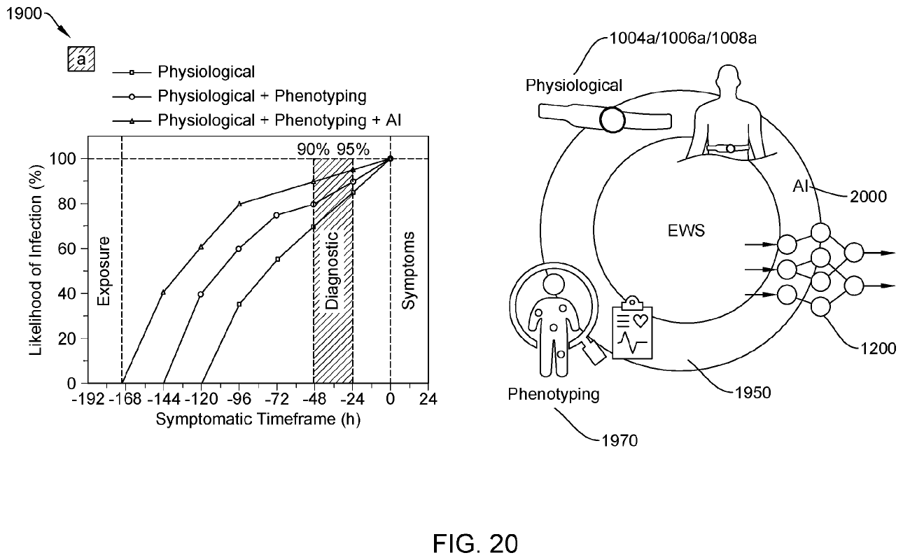

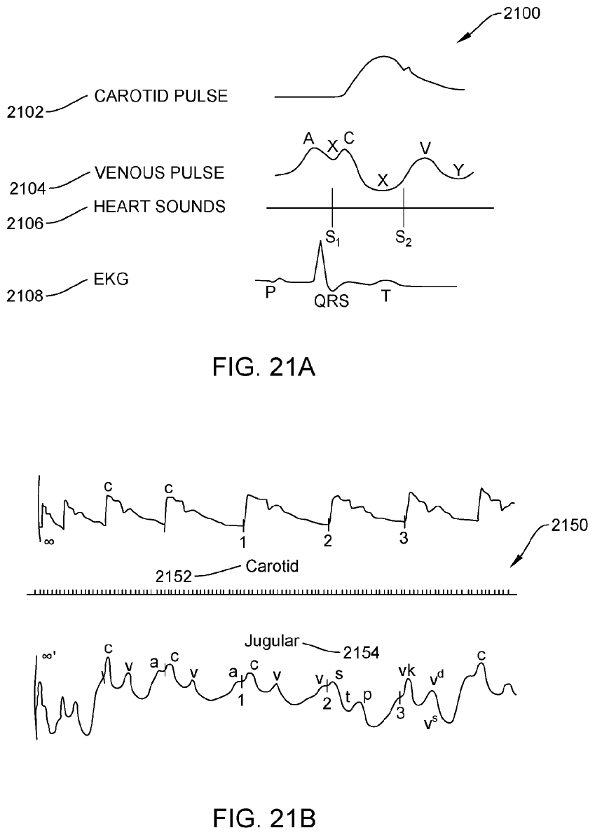

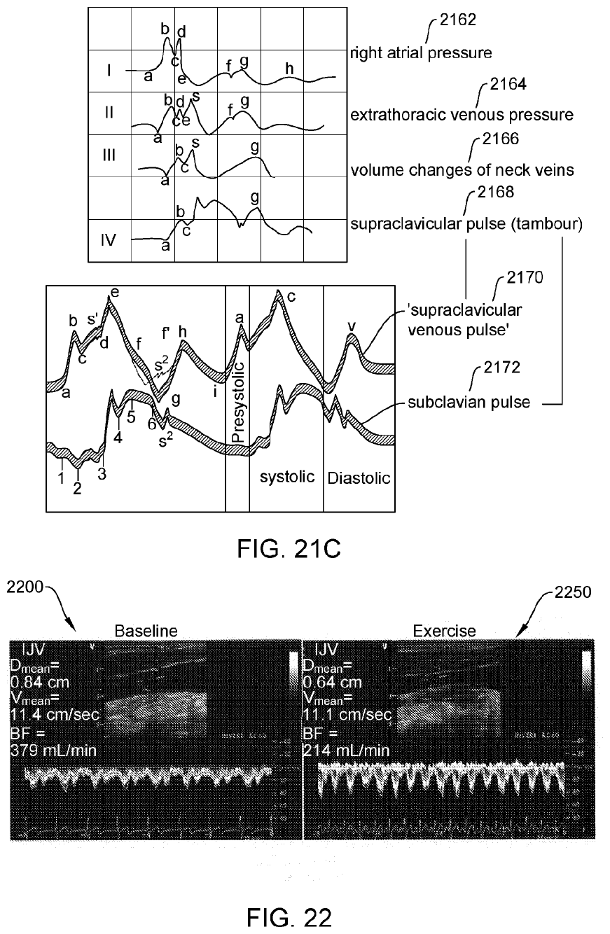

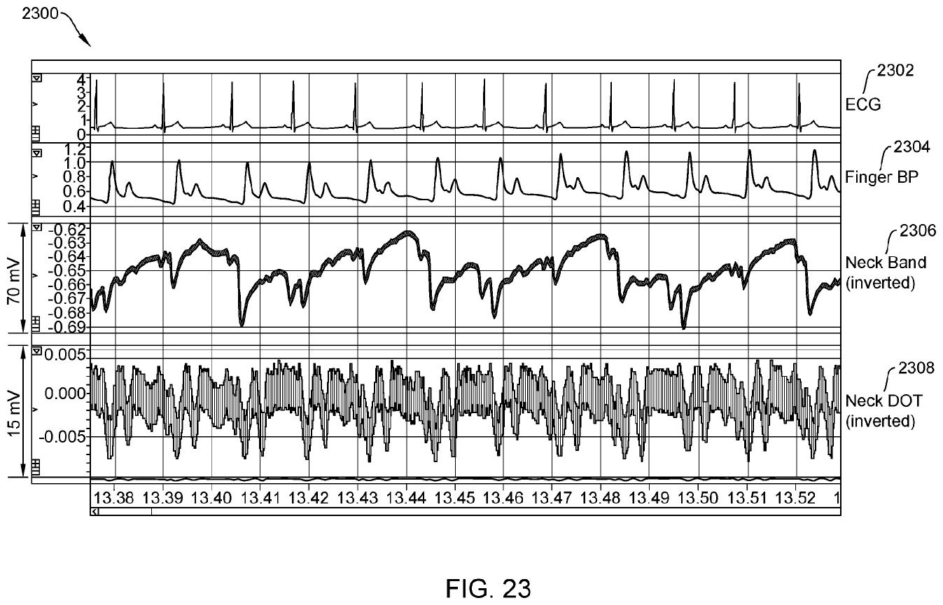

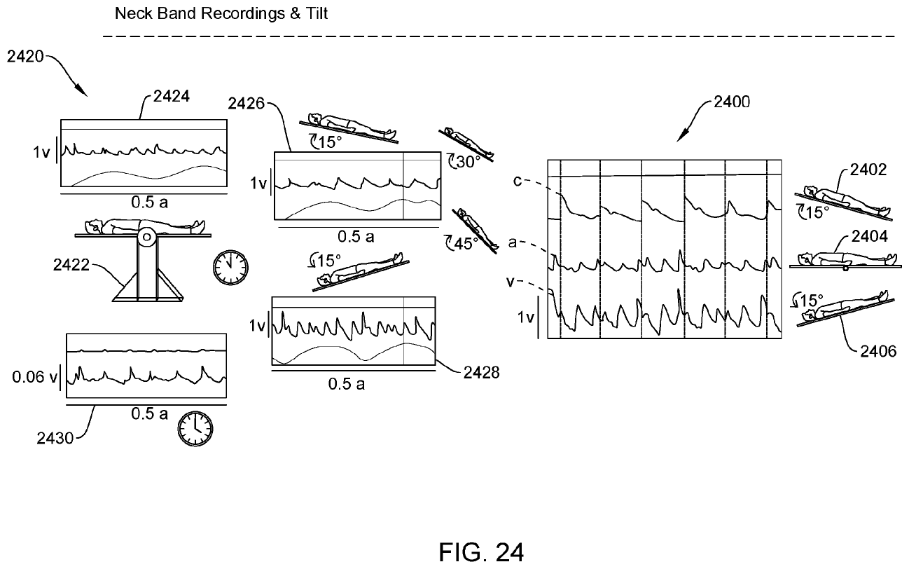

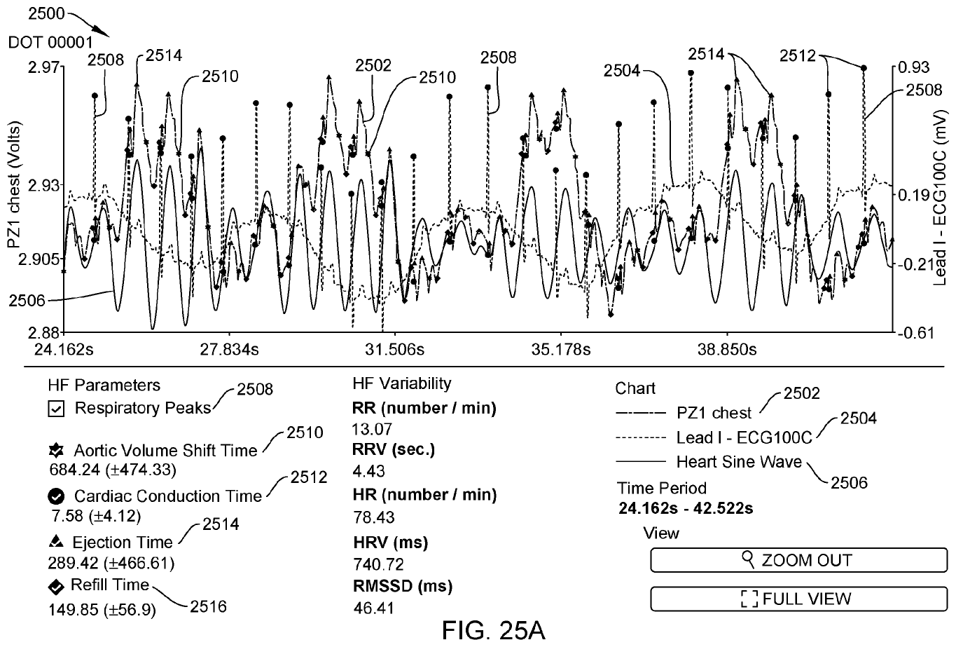

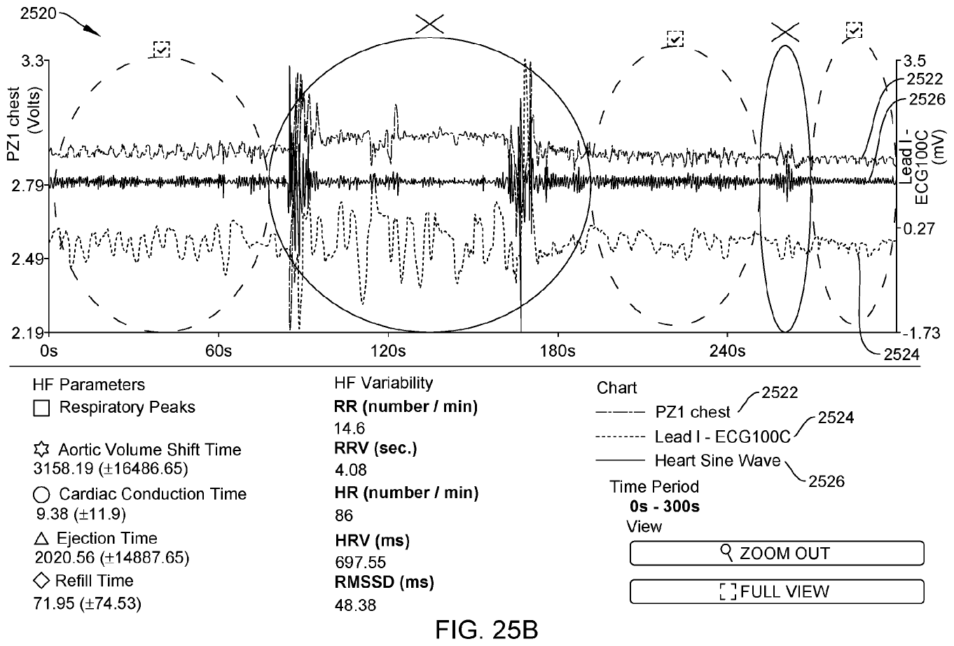

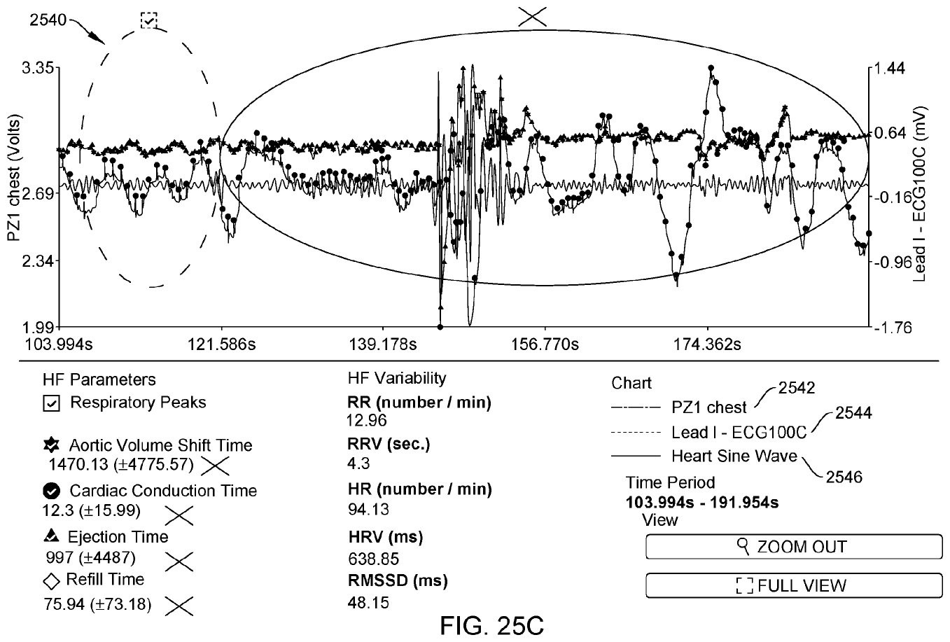

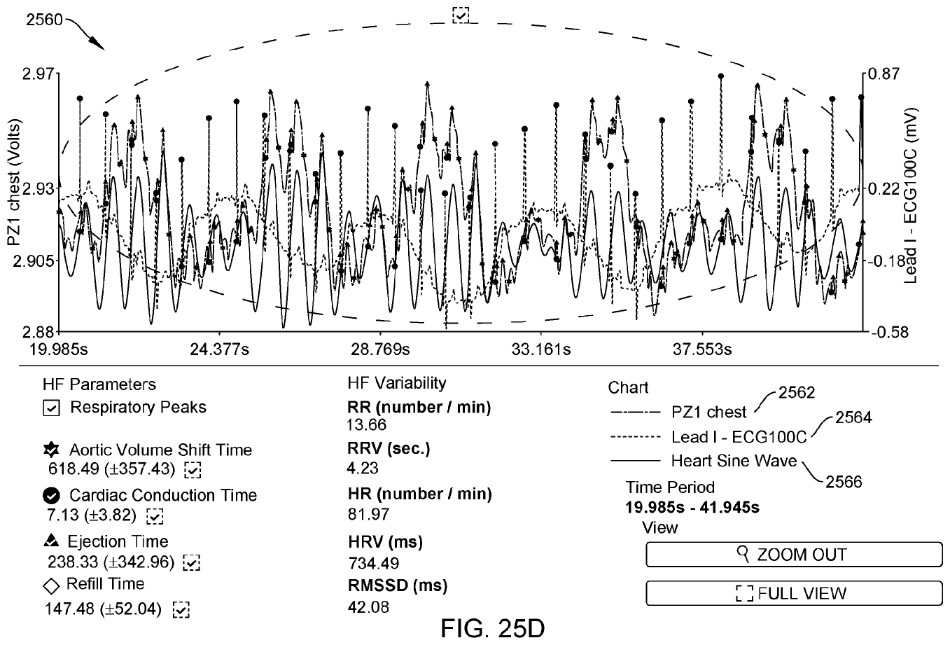

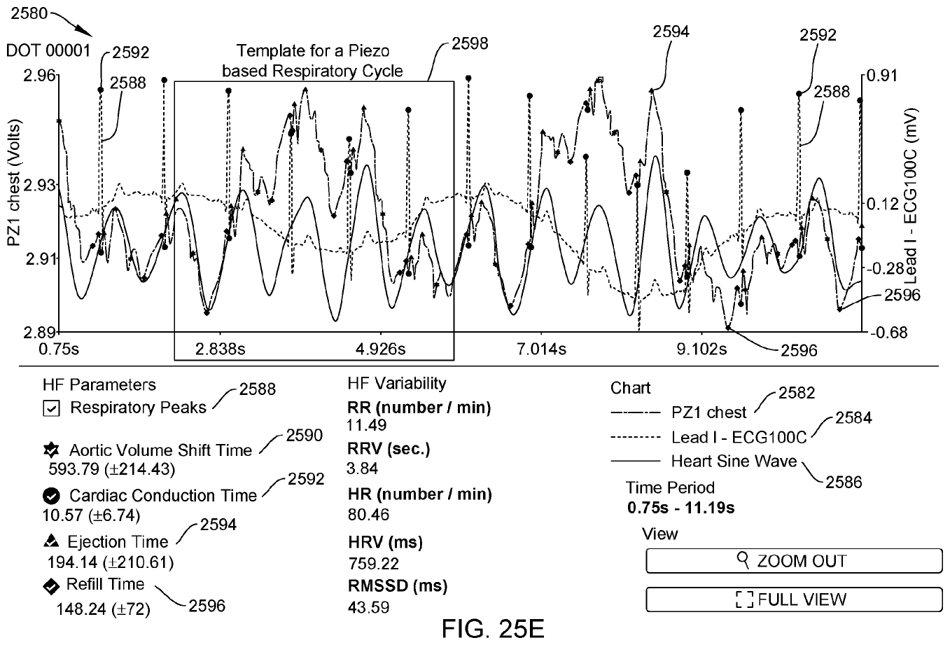

FIG. 3A to 3F illustrates the timing of the ultrasound relative to an electrocardiogram, in which 301 to 306 shows images of the ECG relative to various stages of the heart's mechanical status. FIG. 3A shows the P-wave onset 301, in which all valves are closed 301a, 301b and wherein the atria are refilling with blood. FIG. 3B shows the P-wave body 302, in which the atria are contracting 302a, the atrioventricular valves open 302b. FIG. 3C shows the P-wave offset 303, in which the atria have completed the contraction 303a, the atrioventricular valves close 303b. FIG. 3D shows the QRS onset 304, where blood refilled ventricles start depolarization, wherein relative pressure in the atria-ventricles ensure the valve 304b to be sealed. This can be seen by the first deflection peak on dots which notes the conduction time. FIG. 3E shows the QRS body offset 305, where it shows full ventricle contraction, in which contractility is noted by the large waveform and peak on dots 305c. FIG. 3F shows the QRS-offset or T-wave onset 306, where Aorta valve 306a is shown to be open as blood moves or shifts to the aorta of the heart.

As shown in FIG. 21A, the graph 2100 shows the various morphological features of the carotid pulse 2102 and the jugular venous pulse 2104 (A, X, C, X, V AND Y) relative to S1 and S2 heart sounds 2106 and the P, QRS and T wave in the ECG or EKG 2108, where:
A wave is right atrial contraction;
C wave is early ventricular contraction;
X descent (part 1 and part 2) is downward movement of the ventricle during systolic contraction;
V wave is filling of right atrium;
Y descent is opening of tricuspid valve in diastole.

The timing and amplitude of these morphological changes may change when there are pressure changes in the right atrium.

FIG. 21B shows a graph 2150 having the simultaneous carotid 2152 and venous pulse tracings 2154 taken with a sensitive form of Lombard's tambour. x-x′, relative position of writing points with arcs. This figure illustrates the method of marking c-waves as well as the different terminologies. indicative carotid artery and jugular venous pulse waveforms.

As shown in the top figure of FIG. 21C, the graph 2160 illustrates four curves showing transition of right auricular pulse to supraclavicular venous pulse. I-pressure changes in right auricle 2162; II— pressure changes in intrathoracic veins or extrathoracic venous pressure 2164; III—volume changes of neck vein 2166; IV—supraclavicular pulse taken with tambour 2168. As shown in the bottom figure of FIG. 21C illustrates the supraclavicular venous pulse 2170 (upper) and subclavian pulse 2172 (lower).

FIG. 22 shows jugular venous pulse from an ultrasound probe. The first image shows a baseline JVP 2200 (left image) which highlights a similar morphology to a typical JVP shown in FIG. 3H. The right image shows the JVP with exercise 2250 showing a change in morphology with an increase in amplitude.

FIG. 23 shows the JVP signal 2300 recorded from the neck band sensor 2306 and the neck dot sensor 2308. Both show the typical JVP morphology outlined in textbooks allowing the identification of the A wave corresponding to right atrial contraction, the C wave corresponding to early ventricular contraction, the X descent (part 1 and part 2) corresponding to the downward movement of the ventricle during systolic contraction as well as the V wave which corresponds to filling of the right atrium and also the Y descent corresponding to the opening of tricuspid valve in diastole.

FIG. 24 shows a schematic 2400, 2420 illustrating the change in the detected JVP pulse using the neck band at various tilt table angles 2402, 2404, 2406, 2422 highlighting the value for the sensor to detect JVP at different blood pressures.

While ultrasound can determine timing, the sensor assembly 1002 may be used to measure heart valve signals and determine the blood refill or blood ejection time or period from one or more sensor assemblies 1002. The sensor assembly 1002 may be mounted or placed on the skin over predetermined positions of interest, such as one of the sensor assemblies may be over the heart, to enable continuous and non-invasive monitoring of mechanical events of each cardiac cycle of the person or subject, for example as shown in FIG. 4. While useful information on physiological parameters can be obtained using one sensor assembly 1002, such as identification and duration of each phase of a cardiac cycle 401-408, determining the period at which the heart valves are open and closed, the heart contractility, stroke volume, cardiac output, pulse transit time, and central arterial pressure; further information on physiological parameters can be obtained when a second sensor assembly is coupled to the subject's skin at a different predetermined location compared to the first sensor assembly 1002.

It may be appreciated that the sensor assembly 1002 and its group of sensors (1004, 1006, 1008) are in communication with a processor 1100 with a memory or program 1102 that may allow the processor to execute instructions and store received physiological signals 1004a/1006a/1008a obtained by the sensors 1004/1006/1008 respectively. The processor may have a digital signal processing unit configured for receiving and processing physiological signals from at least one sensor assembly having one or more sensors. The processor 1100 may process the received signals 1004a/1006a/1008a as data values 1004b/1006b/1008b that may be specifically chosen to calculate specific heart function and/or be used to provide an indication of heart health based on the analysed data values. The program or software 1102 of the processor 1100 may be configured to store of measured physiological signals 1004a/1006a/1008a may be processed and may be represented as data values 1004b/1006b/1008b. The information stored may relate to the predetermined position 1004c/1006c/1008c of the sensor assembly 1002, the signals received, the time or period or recording 1004d/1006d/1008d. It may be appreciated that the signals are received at the same time so the signals are synchronised. The stored data 1104 may be allocated as current data 1106 which may be the most recent measurement or historical data 1108. The program or software 1102 with specific algorithms 1110 for identifying relevant predetermined intervals of the received signals 1004a/1006a/1008a for specific measurements as well as being capable of deriving/performing differential analysis or calculating and/or calibrating the signals to generate a data value 1004b/1006b/1008b. The data value 1004b/1006b/1008b may be used to compare and match to a relevant particular set of parameters of the cardiovascular system as shown in Table 2, which may convey the physiological risk of the heart condition or health. The physiological risk in the particular set of parameters may have value range thresholds. It may have a data value range indicating a healthy function and if beyond this predetermined healthy range, it may have a data value range indicating the person to see a clinician. The advantage of storing historical data 1108 is so that this data 1108 may be compared to the current data 1106 which may provide an indication of heart health over time. That is, if the analysis of the data over time is at a worsening condition, while the measurements may be in the healthy range, if the processor projects or predicts that there may be a problem or heart failure, the system will also provide an indication to the person or an alarm to alert the person to see a clinician or to seek further medical advice.

As illustrated in FIG. 4, the ejection and refill time 404, 408a and 408b with other intervals/points can be identified by the ECG electrode 1008 with the waveform displayed 409, the force sensor 1004 with the waveform displayed 411 at the chest, and the displacement sensor 1006 with the waveform displayed 410. The vertical lines traversing through the predetermined periods of signals, now displayed as waveforms (409, 410, 411) from each sensor (1004, 1006, 1008) shows the identified cardiac function 401-408a and b and what is happening during that particular period of time. More specifically, 401 to 408 shows the quantitative timing from various pairs of different morphological features on the signals through a cardiac cycle. 401 shows the atrial contraction time from the piezoelectric sensor signal which in the cardiac cycle corresponds to when the atria contract due to the P wave from the ECG; 402 shows the peak of the R wave of the ECG to a trough in the piezo signal which corresponds to the cardiac conduction time; 403 shows the period at which filled ventricles of fixed volume contract with all valves closed with 1207 myocardial fibers at a given preload and afterload. the closure of the semilunar (aortic and pulmonary) valves. The cardiac isovolumetric contraction 1207 may be known as the tension developed and the velocity of shortening (that is, the ‘strength’) of contraction. The period as indicated by 404 is identified as the ejection period, which may be the time it takes to eject the blood from the heart leading to aortic and pulmonary valve closure at 405. The period as indicated by 407 is identified as the time at which the aortic valve and pulmonic valve are closed and when the tricuspid and mitral valves open at 406 (ie all valves closed. It is called the Isovolumetric Relaxation time. When the AV valves (mitral and tricuspid) open at 406, blood starts refilling the ventricles with 408a and 408b shows the time it may take for the ventricles to refill with blood prior to atrial contraction completing the filling of the ventricles with enough pressure created to close the AV valves. The cardiac cycle then repeats itself.

More physiological signals can be gathered from use of more than one sensor assembly. As shown in FIG. 5, it shows the relationship between the ECG, a first sensor assembly 1002 on the chest, and another sensor assembly 1002 on the arm. The chest signal obtained by these sensor assemblies 1002 may be differentiated 1200 by the processor 1100 to get the actual acoustic sound signal 1202. The processor 1100 may be configured to determine the blood pressure pulse of the subject as shown in Biopac signal or waveform 1204. The cardiac sound signal 1202 may show the region 503 corresponding to the first heart sound or S1 501, which may correspond to the mitral and tricuspid valves closing and corresponds to the pulse. And the second heart sound or S2 502, which may represent the closure of the semilunar (aortic and pulmonary) valves. As the delay of the pulse is known as it reaches the finger (the bottom Biopac signal 1204) of FIG. 5, the program 1102 of the processor 1100 will factor in this natural time delay and can derive the Pulse Transit Time (PTT) 503 based on these received synchronised physiological signals 1004a/1006a/1008a.

In another preferred embodiment, the processor 1100 may be configured to determine the aorta and pulmonary artery valve health from the received physiological signals 1004a/1006a/1008a obtained from placing the first sensor assembly 1002 on the right side of the suprasternal notch and the second sensor assembly 1002 on the left side of the suprasternal notch. As shown in FIG. 6, an example of two sensor assemblies 1002 is being positioned over a subject, in this particular example, the first sensor assembly or first sensor dot may be positioned over the apex 601 and the second sensor assembly or second sensor dot may be positioned over the suprasternal notch 602. These two positions may correspond to the proximity of two key auscultation locations at the 5^{th} intercostal space and above the 2^{nd} intercostal space.

The received synchronised physiological signals 1004a/1006a/1008a from the sensors 1004, 1006, 1008, in which a first sensor assembly 1002 may be over the heart and the second sensor assembly 1002 may be at a different location of the body may allow the processor 1100 to derive or measure the pressure wave amplitude at a predetermined time interval such that data values in relation to the rise/fall time can give the indication of the elasticity of the output vessels or aorta vessels of the subject. The information or data from assessing the relative timing relationships between the aorta vessels and other arteria in the body such as, but not limited to, the iliac crest artery, carotid artery (neck pulse), radial artery (radial pulse) etc. Examples of positioning of the sensor assemblies may be shown in FIGS. 11A to 11E. As the second or more sensors 1002 may be positioned together at any predetermined location over the body, the received physiological signals 1004a/1006a/1008a from each of the sensor assemblies 1002 can be used to plot the full hemodynamic picture of a human being.

As shown in FIGS. 7 and 8, the processor 1100 may derive specific time intervals 1300, 1302 from the received physiological signal of each of the sensor assemblies to plot the central hemodynamic profile of the subject. In particular, the time interval as indicated at C 1300 may correspond to the heart function of blood vessel expansion, in which in the predetermined time interval, the blood vessel may receive the high-pressure pulse. While the time interval as indicated at D 1302 may correspond to the heart function of blood vessel contraction, in which in the predetermined time interval, the pulse may be spread out or spreading out of the pulse. For the chart in which the first sensor assembly may be located over the apex and the second sensor assembly may be positioned over the suprasternal notch, the processor 1100 may derive or calibrate the peak amplitude A 1304 for expansion and B 1306 for contraction at these specific predetermined time intervals. FIG. 8 may show how a processor 1100 may determine the ejection fraction 1308 from deriving or calibrating the amplitudes of 702-A and 702-B from the piezoelectric waveform when the displacement sensors 1006 of the first sensor assembly 1002 and the second sensor assembly 1002 is positioned over or near the apex of the heart 601 and over the suprasternal notch 602 respectively. The processor 1100 may execute the following equation of: Ejection fraction=B as % of A.

Further information by deriving the values of A and B from the piezoelectric waveform 410 may determine the elasticity of the blood vessel. For example, the value from the ratio of A:B may represent the elasticity of the blood vessel, and the value from the ratio: (A/C):(B/D) may be an indication of blood vessel compliance. By comparing the value from the ratio of (A/C):(B/D) at different locations may plot the hemodynamic picture of the human body. The derived value of the ratio of calibrated blood ejection time or calibrated blood refill time may be as accurate and functional compared to ejection fractions measured with ultrasound or other imaging devices used in a clinical setting. The amplitude proportion of the subtracted calibrated piezoelectric waveforms or displacement signals positioned over the apex of the heart 601 and positioned over the suprasternal notch 602 may be correlated with the ejection fraction 1308.

It may be appreciated that the program 1102 or software 1102 for the processor 1100 may use dedicated algorithms 1100 that may synchronise the received physiological signals from each sensor assembly in use, and also depending on where the sensor assembly is positioned over the body, the program may store received physiological signals such that the person can track their heart health progression, which may be vital for early detection of heart disease or the advancement of heart disease.

While the device or apparatus as described may be used, it may also be appreciated that this is also a method or a system for monitoring heart health. It may be appreciated that method steps are required for the processor 1100 comprising the program 1102 to derive data values 1004b/1006b/1008b from received physiological signals 1004a/1006a/1008a from the group of sensors in the sensor assembly 1004/1006/1008 and the software 1102 and algorithms 1110 that allows for the determination of heart function and/or indication of heart health.

For example, in relation to measurement procedures for the determination of cardiac contractility 1207 and estimation of central blood pressure 1208, the device may require at least two sensor assemblies 1002 or two sensor dots 1002 and one or more ECG electrode 1008 (1 lead ECG), in which the first sensor assembly 1002 may be positioned over the cardiac apex 601 and the second sensor assembly may be positioned over the suprasternal notch 602. The processor 1100 may be configured to measure the following parameters: Pulse Transit Time (PTT) 503 or PAT, where the PAT parameter is estimated as the time difference between the R peak of the ECG and a point on the PPG rising edge. The processor 1100 may be configured to calibrate the received physiological signals from the sensor assemblies 1002, wherein the processor 1100 may derive or differentiate 1200 the piezoelectric waveform 410 to determine or estimate ejection fraction 1308. Further, derived values and assessment of timing between valves opening may allow the processor 1100 to derive or calculate the blood refill time 408a and 408b as well as the blood ejection time 404.

For example, the processor 1100 may be configured to precisely estimate ejection fraction 1308 by using the following. Two sensor assemblies 1002 with 1 lead ECG 1008 that could be added for better timing measurements. The ECG lead 1008 may be formed with ECG electrodes 1008 embedded in the sensor assembly 1002 or sensor dot 1002. The sensor dots 1002 may be placed at the cardiac apex 601 and the aortic auscultation position 603. Similarly, the parameters to measure are PTT 503, estimate ejection fraction 1308, and assessment of timing between valves 1310 opening as well as deriving or calculating the blood refill 408a and 408b and blood ejection time 404. By positioning the sensor assemblies 1002 over the cardiac apex 601 and the aortic auscultation position 603, the parameters are more accurate as they target the left heart. Similarly, the same useful measurements could be done for the right heart by moving the second sensor assembly from the aortic auscultation position 603 to the pulmonary valve auscultation position 604.

As the sensor assemblies 1002 can be moved from the over the cardiac apex 601 to any other targeted arterial pulse locations such as the iliac crest artery, radial artery etc, the central hemodynamic picture may be determined. Measurement of pulse elasticity as well as PTT 503 or PAT may be derived from the received physiological signals 1004a/1006a/1008a of the sensor assemblies 1002 to establish peripheral arteria stiffness 1206 and blood pressure 1208. Measurement may be improved when ECG signals 1008a are combined with the force sensor 1004 and the displacement sensor 1006 in the sensor assembly 1002. The measurement may be further improved if the sensor assemblies 1002 or dots 1002 are placed at each peripheral pulse that may be intended to monitor. That may allow for simultaneous monitoring beat by beat. Monitoring heart health and the hemodynamic picture may be vital and important if a person may be on pressure altering medication such as when the person may be in an Intensive Care Unit (ICU).

In another embodiment of the present invention, the processor 1100 is an embedded system 1101 for receiving physiological signals 1103 from the sensor assemblies 1002 or sensor dots 1002. The processor 1100 may comprise a Field Programmable Gate Arrays (FPGA) unit 1105 for configurating the processor 1100 to carry out the aforementioned functions. The embedded system 1101 may have its own display 1107 or touch screen 1107 or an interface 1107 for connecting to an external display 1109 or touch screen 1109 for presenting the graphs or waveforms 409, 410, 411 as shown in FIGS. 4, 5, 7, and 8. The display 1109 and touch screen 1109 may also display the indicator of early detection of heart failure generated by the processor 1100 mentioned in above. The embedded system 1101 may also provide a wired or wireless interface 1111 to connect the sensor assembly 1002 or sensor dot 1002. In one embodiment, the processor 1100 is adapted to generate a triggering signal 1113 at each time interval 1115 to the sensor 1002 or sensor dot 1002. When the sensor 1002 or sensor dot 1002 receives such triggering signals 1113, the sensor 1002 or sensor dot 1002 will make a measurement of the physiological data of the subject. In one embodiment, the embedded system 1113 provides a buffer memory 1117 for each sensor or sensor dot interface. The embedded system 1113 may associate with a server device 1119 for further processing the data 1004a/1006a/1008a received from the sensor or sensor dot 1002. In one embodiment, the embodiment system comprises a Health Level Seven or HL7 protocol stack for formatting the received physiological data before forwarding to a server device 1119.

In another embodiment, the processor 1100 is a computer or smart device 1100 and the method for generating an indicator of early detection of heart failure generated by the processor mentioned in above is implemented as a software application 11002. The method utilised the central processing until 1121 to control one or more set of sensor or sensor dot 1002 for measuring physiological signals of a subject. In one embodiment, the method may select different sets of sensor or sensor dot 1002 for differential analysis measurement 1200. The method is adapted to trigger the measurement of the sensor or sensor dot at different time intervals and collect physiological signals from the sensor or sensor dot 1002 of the present invention to generate an indicator of early detection of heart failure to alert the health professional. The computer or smart device 1100 may connect the sensor or sensor dot 1002 through wired or wireless interface 1123. In one implementation the sensor or sensor dot 1002 is implemented an embedded device 1113 comprising a communication unit 1125 for communicating through a wireless protocol 1127 such as Bluetooth 1129, WIFI 1131, ethernet 1133, 5G 1135, etc.

In one embodiment, the software 1102 is adapted to learn the signal patterns 1137 from historical data 1108 in order to improve the timing for triggering measurement signals 1139 to different set of sensors 1002 or sensor dots 1002. The software 1102 may learn and recognised the pattern 1137 and derive 1139 the measuring timing interval 1141 through artificial intelligent algorithm 1143. The software 1102 or program 1102 may also synchronise received data 1145 from the sensors as graphs or waveforms presented based on timestamps in the data. This information may allow the processor 1100 to derive the time and/or amplitudes corresponding to a particular cardiac function or event, that may be used to calculate heart function such as blood refill time 408a and 408b and blood ejection time 404, vessel contractility 1207 and vessel expansion 1209 etc.

In another embodiment of the present invention, there is provided a method of generating an indicator of early detection of heart failure comprising the steps of:

Step 1: Selecting sensor type, location and configuration (as shown in FIGS. 10A and 10B, 10C and Table 1.)

As shown in the table in FIG. 10A 1400, the identification of group of sensor(s) are attributed to a sensor ID 1402. When the signals are obtained, depending on the signals sensed by the sensor assembly/assemblies, the specific sensor ID/type may be attributed. As shown in FIGS. 10B and Figure legend 10C, the each of the sensor assemblies (sensors 1 to 10) 1002, may be chosen. For the sensor assemblies 6 to 10, it shows that the ECG electrode 1008 is embedded in the sensor assembly. It may be appreciated that the sensor assemblies 2 to 5, 7 to 10 may have a front FSR 1004 and a back FSR 1004 component.

For example, in one embodiment:

Sensor ID number 1 is attributed when signals from a force sensor 1404 and a displacement sensor 1406 are sensed/transferred.

Sensor ID number 2 is attributed when signals from a first force sensor 1404, a displacement sensor 1406, a second force sensor 1408 that can calibrate the first force sensor 1404 are sensed/transferred.

Sensor ID number 3 is attributed when signals from a first force sensor 1404, a displacement sensor 1406, a second force sensor 1408 that can calibrate the first force sensor 1404, and disconnected FSR 1410 are sensed/transferred.

Sensor ID number 4 is attributed when signals from a first force sensor 1404, a displacement sensor 1406, a second force sensor 1408 that can calibrate the first force sensor 1404, disconnected FSR 1410, and a first respiratory band 1412 are sensed/transferred.

Sensor ID number 5 is attributed when signals from a first force sensor 1404, a displacement sensor 1406, a second force sensor 1408 that can calibrate the first force sensor 1404, disconnected FSR 1410, and a first and second respiratory band 1412, 1414 are sensed/downloaded.

Sensor ID number 6 is attributed when signals from a first force sensor 1404, a displacement sensor 1406, and an ECG electrode/1 lead ECG 1416 are sensed/transferred.

Sensor ID number 7 is attributed when signals from a first force sensor 1404, a displacement sensor 1406, and an ECG electrode/1 lead ECG 1416, a second force sensor 1408 that can calibrate the first force sensor 1404 are sensed/transferred.

Sensor ID number 8 is attributed when signals from a first force sensor 1404, a displacement sensor 1406, and an ECG electrode/1 lead ECG 1416, a second force sensor 1408 that can calibrate the first force sensor 1404, and a disconnected FSR 1410 are sensed/transferred.

Sensor ID number 9 is attributed when signals from a first force sensor 1404, a displacement sensor 1406, and an ECG electrode/1 lead ECG 1416, a second force sensor 1408 that can calibrate the first force sensor 1404, a disconnected FSR 1410, a first respiratory band 1412 are sensed/transferred.

Sensor ID number 10 is attributed when signals from a first force sensor 1404, a displacement sensor 1406, and an ECG electrode/1 lead ECG 1416, a second force sensor 1408 that can calibrate the first force sensor 1404, a disconnected FSR 1410, and a first and second respiratory band 1412, 1414 are sensed/transferred.

It may be appreciated that ‘disconnected’ in the term disconnected FSR may be defined as not mechanically connected to the front sensors—‘mechnically isolated’ so that the signals from the disconnected sensor only come from the finger/wrist rather than the chest.

Step 2: Selecting a group of biometrics to measure

As shown in FIGS. 11A to 11E, the location for various sensor assembly positions may be positioned over following areas of the body:

As shown in FIG. 11A, the following chest locations may be selected for the placement of the one or more sensor assemblies 1002: The cardiac apex (position T) 601, the suprasternal notch (position S) 602, aortic valve (position A) 606, pulmonary valve (position P) 607, Mitral valve (position M).

As shown in FIG. 11B, the following hand and wrist locations may be selected for the placement of the one or more sensor assemblies: The wrist (position R) 609, and the finger (position F) 610;

As shown in FIG. 11C, the following neck locations may be selected for the placement of the one or more sensor assemblies: The carotid (position C) 611;

As shown in FIG. 11D, the following pelvic locations may be selected for the placement of the one or more sensor assemblies: The iliac crest (position I) 612;

As shown in FIG. 11E, the following femoral to ankle locations may be selected for the placement of the one or more sensor assemblies: The femoral or groin (position F) 613, the popliteal or back of the knee (position P) 614, posterior tibial or ankle (position PT) 615.

In one embodiment, the physiological signals sensed in the listed locations or positions may obtain a central and peripheral hemodynamic picture of the human body.

It may be appreciated that the sensors may also be positioned over other parts of the body that are not listed to obtain a further comprehensive hemodynamic picture of the human body as more signal or information are received to be analysed.

Table 1 shows the minimum number of sensors needed as well as the Sensors needed to obtain specific biometrics or the subject.

| Minumun | Sensors needed | | | | | | | |
|---|---|---|---|---|---|---|---|---|
| Number | (sensor ID as | Minimum | | | | | | |
| of sensors | per FIGS. 10A | Positions | | | | | | |
| needed | and 10B, 10C) | needed | Biometrics category | Specific Biometric | Duration | Amplitude | Area | Slope |
| 1 | 1 to 10 | Apex | Cardiac | 1^{st} Cardiac sound | X | X | | |
| | | | Electromechanics | | | | | |
| 1 | 1 to 10 | Apex | Cardiac | 2^{nd} Cardiac sound | X | X | | |
| | | | Electromechanics | | | | | |
| 1 | 1 to 10 | Apex | Cardiac | 3^{rd} Cardiac sound | X | X | | |
| | | | Electromechanics | | | | | |
| 1 | 1 to 10 | Apex | Cardiac | Refill time | X | | | |
| | | | Electromechanics | | | | | |
| 1 | 6 to 10 | Apex | Cardiac | Cardiac | X | | | |
| | | | Electromechanics | Conduction Time | | | | |
| 1 | 1 to 10 | Apex | Cardiac | Ejection time | X | | | |
| | | | Electromechanics | | | | | |
| 1 | 1 to 10 | Apex | Cardiac | Cardiac Output | X | | | |
| | | | Electromechanics | Valves | | | | |
| 1 | 1 to 10 | Apex | Cardiac | Aortic Volume | X | | | |
| | | | Electromechanics | shift | | | | |
| 1 | 1 to 10 | Apex | Cardiac | Cardiac Output | X | | | |
| | | | Electromechanics | Valves | | | | |
| 1 | 6 to 10 | Apex | Cardiac | Pre ejection | X | | | |
| | | | Electromechanics | period | | | | |
| 1 | 1 to 10 | Apex | Cardiac | Heart Rate | X | | | |
| | | | Electromechanics | Variability | | | | |
| 1 | 1 to 10 | Nect over | Cardiac | Jugular Venous | X | X | X | X |
| | | jugular vein | electromechanics | Pulse | | | | |
| | | | | corresponding | | | | |
| | | | | to right atrial | | | | |
| | | | | pressures | | | | |
| 2 | 1 to 10 | Apex, | Cardiac | C. Vessel | X | | | |
| | | SSN/ | Electromechanics | expansion | | | | |
| | | Aortic/ | | receiving the | | | | |
| | | Pulmonary | | high pressure | | | | |
| | | | | pulse | | | | |
| 2 | 1 to 10 | Apex, | Cardiac | D. Vessel | X | | | |
| | | SSN/ | Electromechanics | contraction | | | | |
| | | Aortic/ | | spreading out | | | | |
| | | Pulmonary | | the pulse | | | | |
| 2 | 1 to 10 | Apex, | Cardiac | A. Calibrated | | X | | |
| | | SSN/ | Electromechanics | peak amplitude | | | | |
| | | Aortic/ | | for expansion | | | | |
| | | Pulmonary | | | | | | |
| 2 | 1 to 10 | Apex, | Cardiac | B. Calibrated | | X | | |
| | | SSN/ | Electromechanics | peak amplitude | | | | |
| | | Aortic/ | | for contraction | | | | |
| | | Pulmonary | | | | | | |
| 2 | 6 to 10 | Apex, | Central | Contractility | X | | | |
| | | Aortic Valve | haemodynamic left | (PAT between | | | | |
| | | | heart | Morphic Dots | | | | |
| | | | | and ECG) (Apex | | | | |
| | | | | to aortic valve | | | | |
| | | | | auscultation) (LH) | | | | |
| 2 | 2 to 5, 7 to 10 | Apex, | Central | Ejection Fraction | | X | | |
| | | Aortic Valve | haemodynamic left | Calibrated DOTs | | | | |
| | | | heart | differentiated | | | | |
| | | | | (Apex to aortic | | | | |
| | | | | valve | | | | |
| | | | | auscultation) (LH) | | | | |
| 2 | 6 to 10 | Apex, | Central | Contractility | X | | | |
| | | Pulmonary | haemodynamic right | (PAT between | | | | |
| | | valve | heart | Morphic Dots | | | | |
| | | | | and ECG) (Apex | | | | |
| | | | | to pulmonary valve | | | | |
| | | | | auscultation) (RH) | | | | |
| 2 | 2 to 6, 7 to 10 | Apex, | Central | Ejection Fraction | | X | | |
| | | Pulmonary | haemodynamic right | Calibrated DOTs | | | | |
| | | valve | heart | differentiated | | | | |
| | | | | (Apex to | | | | |
| | | | | pulmonary valve | | | | |
| | | | | auscultation) (RH) | | | | |
| 2 | 6 to 10 | Apex, | Central | Central PAT | X | | | |
| | | SSN/ | Haemodynamics | | | | | |
| | | Aortic/ | | | | | | |
| | | Pulmonary | | | | | | |
| 2 | 6 to 10 | Apex, | Central | Central PTT | X | | | |
| | | SSN/ | Haemodynamics | | | | | |
| | | Aortic/ | | | | | | |
| | | Pulmonary | | | | | | |
| 2 | 6 to 10 | Apex, | Central | Central PEP | X | | | |
| | | SSN/ | Haemodynamics | | | | | |
| | | Aortic/ | | | | | | |
| | | Pulmonary | | | | | | |
| 1 | 2 to 5, 7 to 10 | Apex, | Central | Central BP | | X | | |
| | | SSN/ | Haemodynamics | | | | | |
| | | Aortic/ | | | | | | |
| | | Pulmonary | | | | | | |
| 2 | 6 to 10 | apex/SSN/ | Peripheral | PAT Cardiac to | X | | | |
| | | Aortic/ | Haemodynamics | finger | | | | |
| | | Pulmonary, | | | | | | |
| | | finger | | | | | | |
| 2 | 6 to 10 | apex/SSN/ | Peripheral | PTT Cardiac to | X | | | |
| | | Aortic/ | Haemodynamics | finger | | | | |
| | | Pulmonary, | | | | | | |
| | | finger | | | | | | |
| 2 | 6 to 10 | apex/SSN/ | Peripheral | PEP Cardiac to | X | | | |
| | | Aortic/ | Haemodynamics | finger | | | | |
| | | Pulmonary, | | | | | | |
| | | finger | | | | | | |
| 1 | 2 to 5, 7 to 10 | apex/SSN/ | Peripheral | Peripheral BP | | X | | |
| | | Aortic/ | Haemodynamics | finger | | | | |
| | | Pulmonary, | | | | | | |
| | | finger | | | | | | |
| 2 | 6 to 10 | apex/SSN/ | Peripheral | PAT Cardiac to | X | | | |
| | | Aortic/ | Haemodynamics | wrist (radial) | | | | |
| | | Pulmonary, | | | | | | |
| | | wrist | | | | | | |
| 2 | 6 to 10 | apex/SSN/ | Peripheral | PAT Cardiac to | X | | | |
| | | Aortic/ | Haemodynamics | wrist (radial) | | | | |
| | | Pulmonary, | | | | | | |
| | | wrist | | | | | | |
| 2 | 6 to 10 | apex/SSN/ | Peripheral | PAT Cardiac to | X | | | |
| | | Aortic/ | Haemodynamics | wrist (radial) | | | | |
| | | Pulmonary, | | | | | | |
| | | wrist | | | | | | |
| 1 | 2 to 5, 7 to 10 | apex/SSN/ | Peripheral | Peripheral BP | | X | | |
| | | Aortic/ | Haemodynamics | radial | | | | |
| | | Pulmonary, | | | | | | |
| | | wrist | | | | | | |
| 2 | 6 to 10 | apex/SSN/ | Peripheral | PAT Cardiac to | X | | | |
| | | Aortic/ | Haemodynamics | iliac | | | | |
| | | Pulmonary, | | | | | | |
| | | iliac | | | | | | |
| 2 | 6 to 10 | apex/SSN/ | Peripheral | PAT Cardiac to | X | | | |
| | | Aortic/ | Haemodynamics | iliac | | | | |
| | | Pulmonary, | | | | | | |
| | | iliac | | | | | | |
| 2 | 6 to 10 | apex/SSN/ | Peripheral | PAT Cardiac to | X | | | |
| | | Aortic/ | Haemodynamics | iliac | | | | |
| | | Pulmonary, | | | | | | |
| | | iliac | | | | | | |
| 2 | 3 to 5, 7 to 10 | apex/SSN/ | Peripheral | Peripheral BP | | X | | |
| | | Aortic/ | Haemodynamics | iliac | | | | |
| | | Pulmonary, | | | | | | |
| | | iliac | | | | | | |
| 1 | 4, 5, 9, 10 | Apex | Cardiorespiratory | Change in tidal | X | X | X | |
| | | | mechanics | volume | | | | |
| 2 | 5, 10 | Apex, | Cardiorespiratory | respiratory effort | X | X | X | |
| | | abdominal band | mechanics | | | | | |

The minimum positions need in the column of Table 1 may be: Apex, suprasternal notch (SN or SSN), aortic valve (AV), pulmonary valve (PV), Radial, Iliac, finger, carotid. In one embodiment, the peripheral haemodynamics can be measured from heart to arteries in the leg (such as femoral, popliteal or tibial). In addition, vascular haemodynamics can be measured between any two arterial positions.

Step 3: Synchronising and receiving signal data or raw signal data from the sensor assemblies so that the waveforms 409, 410, 411 line up with respect to time (as also shown in FIG. 12) in which all waveforms are acquired at the same time.

Step 4: Amplitude calibrating so the amplitude of the waveforms can be derived (as shown in FIG. 13)

Step 5: The processor will receive the data and preform preprocessing of the data such as magnification, noise filtering, normalisation, etc. The program may be configured to map the waveforms associated with signals received from the specific sensor in the sensor assembly.

Step 6: Once the signal has been transformed or digitised, the signal is uploaded to the cloud or server and downloaded by the processor in real time. At which the processor is configured to select and display data from the cloud in real time. The data may be data from current measurements or historical measurements (see Table 2 below)

Table 2 shows example reference thresholds of parameters by the system which may determine the patient's risk of heart failure.

TABLE 2

| | | Green | Amber | Red |
|---|---|---|---|---|
| Uncontrollable risk | Family history of heart failure | No | | Yes |
| factors | Gender | Female | | Male |
| | Age | <50 | 50-70 | >70 |
| | family history of cardiomyopathy | No | | Yes |
| | history of rheumatic fever | No | | Yes |
| | history of alcohol abuse | No | | Yes |
| | history of taking drugs that can damage | No | | Yes |
| | the heart muscle, such as some cancer | No | | Yes |
| | drugs | | | |
| Controllable risk | Smoking | No | | Yes |
| factors | alcohol binging (number of times have | 0 | 1-3 | >3 |
| | more than 3 drinks an hour in a week) | | | |
| | Diet (Rating out of 10 based on | 8-10 | 4-7 | <4 |
| | diversified, balanced and healthy) | >120 | 30-120 | <30 |
| | Exercise at least 150 minutes of | | | |
| | moderate aerobic activity or 75 minutes | | | |
| | of vigorous aerobic activity a week | | | |
| | Weight (BMI range) | <25 | 25-30 | >30 |
| Symptoms | Shortness of breath with activity or when | No | | Yes |
| | lying down | | | |
| | Fatigue and weakness | No | | Yes |
| | Swelling in the legs, ankles and feet | No | | Yes |
| | Rapid or irregular heartbeat | No | | Yes |
| | Reduced ability to exercise | No | | Yes |
| | White to yellow coated tongue | No | | Yes |
| | Persistent cough or wheezing with white | No | | Yes |
| | or pink blood-tinged mucus | | | |
| | Swelling of the belly area (abdomen) | No | | Yes |
| | Nausea and lack of appetite | No | | Yes |
| | Difficulty concentrating or decreased | No | | Yes |
| | alertness | | | |
| | Chest pain if heart failure is caused by a | No | | Yes |
| | heart attack | | | |
| | systolic left ventricular dysfunction via | >55 | 40-55 | <40 |
| | an echocardiogram (echo) but | | | |
| | asymptomatic that shows an ejection | | | |
| | fraction (EF) <= 40% | | | |
| Risk factors from | Resting Heart rate | <90 | 90-110 | >110 |
| monitoring from | Heart rate variability (ms) | >100 | 50-100 | <50 |
| patient managing use | Respiratory rate | <25 | 25-30 | >30 |
| of device | Dyspnea (shortness of breath) with | None | Minor | Significant |
| | exercise | | | |
| | Blood pressure (systolic) | <140 | 140-170 | >170 |

From Table 2, in one implementation, a questionnaire regarding uncontrollable risk factors may be entered by the person or the person's clinician. In another embodiment, the risk factors are generated in the processor. In another embodiment, the processor may fetch a list from a server and personalised by preliminary signals obtained from the sensors. Such uncontrollable risk factors may assist clinicians to flag/note persons of higher risk or propensity to have heart failure. Based on the result value of the risk generated, care and frequency of monitoring may be generated and recommended/required for persons categorised as high risk. Uncontrollable risk factors may include: family history of heart failure, gender, age, family history of cardiomyopathy, any history of rheumatic fever, any history of alcohol abuse, any history of drugs or medication that can damage the heart muscle (for example some cancer drugs).

Controllable risk factors are mainly parameters associated with a person's lifestyle and/or diet. Such controllable factors may be: Smoking, alcohol binging (number of times have more than 3 drinks an hour in a week), diet (rating out of 10 based on diversified, balanced and healthy), Exercise time, and weight (BMI Range).

As a possible indication of heart failure, the patient may have any of the following symptoms: Shortness of breath with activity or when lying down, fatigue and weakness, swelling in the legs, ankles and feet, rapid or irregular heartbeat, reduced ability to exercise, persistent cough or wheezing with white or pink blood-tinged mucus, swelling of the belly area (abdomen), nausea and lack of appetite, difficulty concentrating or decreased alertness, chest pain if heart failure is caused by a heart attack. If “Yes” to any of the above symptoms, then the program may categorise the patient as ‘Red’.

Risk factors from monitoring from patient managing use of device may determine the following parameters of interest: resting heart rate, heart rate variability (ms), respiratory rate, Dyspnea (shortness of breath) with exercise, blood pressure (systolic), very rapid weight gain from fluid buildup over a short period of time eg. A month (kg).

Heart Failure test metrics from clinician managing use of device of the present invention may determine the following parameters of interest: heart sounds (eg. 3^{rd} sound), lung sounds (wheezing or crackles), pulse transit time from base heart to top, cardiac conduction times, valve actions, ejection time, refill times, contractility, elasticity, vessel compliance, and hemodynamic function.

Depending on the answers of the questionnaire of one embodiment, the algorithm of the program may categorise the risk value into three or more groups or sets of parameters: such as Green, amber, and red. Green being low risk, amber being moderate risk, and red being high risk of heart failure. It may be appreciated that these thresholds may have in-between parameters close to the boundaries of the thresholds which may assist where it may indicate an upper moderate risk or a lower moderate risk. In another embodiment, the risk value may be a continuous value or even an equation for further calculation.

It may be appreciated that when at least one uncontrollable risk factor is red, regardless of other factors being green or amber, the person is to be monitored in accordance of high risk of heart failure protocols.

Step 7: Comparing to example/reference signal. The marked waveform is shown in FIG. 4 where the lines indicate certain morphological features through a person's cardiac cycle. And the example/reference signal may have the same or similar waveform as FIG. 4 (not shown).

Step 8: Measuring test signals based on example/reference signals. An example of test signals may be shown in FIG. 4, where the processor may retrieve example/reference signals similar or exact waveform and predetermined time intervals as FIG. 4 to conduct measuring.

Step 9: Calculating values of key metrics based on calibration. The value in the table automatically varies according to the spacing relative to the calibration. Example values are shown in FIG. 9.

Step 10: Mapping or graphing parameters over time. For example, the graph of the left ventricular ejection time 1500 is shown in FIG. 14.

Step 11: Preparing a care plan 1600 which may be updated in real time based on measurements/signals received from the sensor assembly/assemblies 1002.

For example, a customised care plan 1600 may be summarised in the table as shown in FIG. 15.

Step 12a: Differentiating 1200 displacement sensor signals/piezoelectric sensor signals for determining/deriving cardiac sounds at predetermined time interval. For example, the mapped cardiac sounds with differentiated piezo signal is shown in FIG. 5. By deriving the interval between the 1^{st} Cardiac Sound (S1) 501 and the 2^{nd} Cardiac Sound (S2) 502, the Pulse Transit Time (PTT) 503 can be determined.

Step 12b: Estimating central pressure based on PTT 503 from cardiac apex 601 to suprasternal notch 602, for example when the sensor assemblies are placed over these two locations (as shown in FIG. 16).

Step 12c: Predetermined time intervals of the waveforms may be used to plot the central hemodynamic profile. As shown in FIG. 7, time interval C 1300 corresponds to the Vessel expansion receiving the high-pressure pulse, while time interval D 1302 corresponds to the Vessel contraction spreading out the pulse. The waveforms generated from the signals received from the FSR apex and the FSR suprasternal notch may have the peak amplitudes at predetermined time intervals calibrated. The program 1102 may determine in a region above the actual signal—displaying a demonstration signal with the vertical lines in place displaying measurement locations for each parameter. This could be the image from the patient's last recording. As shown in FIG. 7, ‘A’ 1304 corresponds to the calibrated peak amplitude for expansion, while ‘B’ 1306 corresponds to peak amplitude for contraction. The program 1102 may determine or derive the elasticity of the vessel by taking the ratio of A:B, and the program may determine or derive an indication of vessel compliance by taking the ratio of (A/C):(B/D). When comparing ratio (A/C):(B/D) at different locations can plot the hemodynamic picture for the patient or clinician that is responsible for the patient. The processor 1100 or program 1102 may be configured to derive 1200 amplitude proportion of the subtracted calibrated piezo waveforms of Suprasternal Notch and cardiac apex (SN-Apex), in which the amplitude proportion may be correlated with the ejection fraction 1308. The program 1102 may also have a notification means 1700 to the patient or user. When notifying the patient to see a clinician either by alert from the system or for a regular check-up, the clinician may measure the ratio of calibrated ejection time/refill time with other verification methods such as ultrasound or other image devices to verify the results determined through use of the device with the sensor assemblies.

Step 12d: Calculating ejection fraction 1308. The program 1102 may move the pair of vertical measurement lines to each of the locations as per the demo and take measurement. As shown in FIG. 8, the derived/calibrated SN-Apex piezo waveform may be used to determine the ejection fraction 1308. Once the program 1102 derives 1200 the data values of A and B, the ejection fraction 1308 can be calculated with the formula: Ejection Fraction=B as % of A.

Step 12e: Calibrating waveforms for blood pressure estimate. As shown in FIG. 17, an example showing the predetermined peaks of the pressure waveform 4707 from the piezo sensor 1006, and the pressure waveform 4708 from FSR 1004, obtained with a single point calibration 1210 can be compared with beat by beat non-invasive blood pressure measurement 4709.

Step 12f: Determining tissue compliance 4809. As shown in FIG. 18, an example of different tissue compliance such as the chest (top waveform) vs the wrist (bottom waveform) is shown. The compliance may be measured by the average Fb minus average Fc 4807, 4808.

Another step that may be utilised by the program may be to determine cardiac function or parts such as:

Selecting the predetermined placement of the sensor assemblies over the identified areas of the subject as shown in FIG. 6 may allow the sensors to obtain information relating to the following valves:

Aortic valve 1900: the S2 components of the heart sound; and determining the time interval at which the semi lunar valve (aortic and pulmonary) are closed.

Pulmonary valve 1902: the S1 components of the heart sound

Tricuspid valve 1904: the time interval at which the tricuspid valve closes; and the S1 heart sound signal can be received.

Mitral valve 1906: the time interval at which the mitral valve (M1) closes; the S1 heart sound is louder than S2; Lub and dub sounds are received—wherein when the sensor is placed over a child's mitral valve instead of an adult, there may be a S3 or third cardiac sound, which may be normally heard in children. For example, instead of lub dub for adults, it may be heard as a lub dub dub; S4 may be heard as just S1 (late diastolic); and S4 may be low pitched or gallop-sounding.

Another step that may be utilised by the program 1102 may be to determine when the waveform may be derived based on reference signals that allows the program to compare and match so that relevant predetermined intervals, such as amplitude or time intervals can be calibrated or calculated or derived 1200. The program 1102 may Freeze the lines and the value when in matched with the referenced position.

Another step that may be utilised by the program 1102 may be that once all values are measured, with the data entered, the data may be loaded to the cloud or a server or a storage medium 1170 These data may be categorised as historical data values 1108 and may be retrieved by the program 1102 for comparison with current data 1106 or measurements to determine whether a particular parameter or factor is worsening over time.

Another step that may be utilised by the program 1102 may be that a graph can be mapped or displayed for each parameter including historical values over time.

Another step that may be utilised by the program 1102 may be that actions from care plan 1600 can be loaded on the same graph eg medication, exercise similar to Table 1. The care plan 1600 is dynamically updated depending on the current measurements or data values 1106 based on an improved or a relative worse measurement compared to the previous use of the sensor assemblies 1002. It may be appreciated that the care plan can also be updated by a clinician manually.

Once the sensor assemblies 1002 are coupled to the patient at other predetermined locations, the processor may repeat Steps 1 to 12f as well as other mentioned steps, if applicable.

In another preferred embodiment of the present invention, the processor 1100 may utilise an artificial intelligence (AI) software 1102 or computer program 1102 which may be configured to learn various data patterns and insights.

FIG. 25A shows an image of a computer screen of sensor signals 2500 that are automatically annotated for various morphological features that correspond to key cardiorespiratory events. These features are then used to calculate relative timings, amplitudes, slopes or areas that correspond to key cardiorespiratory parameters. Examples of these are displayed underneath including respiratory rate 2508, aortic volume shift 2510, cardiac conduction time 2512, ejection time 2514 and refill time 2516. Also shown are heart rate and heart rate variability. Other parameters that can be added include change in tidal volume, respiratory effort, ejection fraction, blood pressure, pulse transit time, pulse wave velocity, contractility and pre ejection period.

FIG. 25B shows a screen 2520 with ovals indicated on the graph that highlights artefacts to be rejected that reduce the measurement of variances for various calculated parameters. These are shown as crosses corresponding to the specific features. This process is applicable to any of the cardiorespiratory features that can be detected as per FIG. 25A. This process can be performed using a manual process or an automatic process using software from a processor.

FIG. 25C shows a screen 2540 and indicative method for recalculating various parameters after artefact rejection as in FIG. 25B. Once the artefacts are rejected, a method is used that replaces the feature with a value that fits into the duration of the signals being used for analysis. This could be halfway between the feature before and the feature after (in the case of only one feature in a row being rejected) or multiple features being replaced by a number equal to the number being replaced—for an example requiring duration calculations, in the case of 5 in a row being rejected, 20% of the duration between the feature before and after. Another method may be replacing each one with the average value. Once this artefact rejection replacement has been performed, the new average and measurement of variance can be recalculated with the resulting measurement of variance being reduced (this is also displaced in the figure shown in 25A and 25B).

FIG. 25D shows a screen 2560 of a computer base analytical software system which automatically eliminates the artefacts part of the signal before estimating measurement of variances. The removal of artefacts reduces the measurement of variances of cardiac parametric calculations. The measurement of variance of the key cardiac parameters, namely, Aortic Volume Shift, cardiac conduction time, ejection time etc. are within clinically accepted ranges, hence the sensory data and the associated analytics are proven to be accepted by the clinician.

As shown in FIG. 25E, FIG. 25E shows a screen 2580 of a computer base analytical software system, which displays the effects of artefacts on the cardiac parametric calculation in the real-life sensory signals, after automatically removing significant artefacts. Rapid template matching using machine learning is advantageous to eliminate artefacts automatically. This example figure showcases a selected template 2598 as a gold standard respiratory cycle based on piezo sensor. This template 2598 is used to score each of the recorded respiratory cycles based on similarity. The higher the similarity score represents a better match, hence indication of a good cycle, in which the data can be accepted clinically. A lower similarity score on the other hand indicates potential artefacts or other kinds of noisy signal and cycle.

As shown in FIG. 25F, FIG. 25F shows a screen 2600 of a computer based analytical software system, which displays an example of the cardiac parametric calculation in the real-life sensory signals, which depicts the methodology of the automatically rejecting artefacts cycles within the recorded time series signal. Matching analysis based on machine learning or a machine learning engine involves an advanced motif template matching, using angular positions of multi-feature points, within the time period defined by a respiratory cycle. This automatic visual approach mimics a cognitive interactive method to match and eliminate artefacts. Similar score is a predictive outcome of a trained learning algorithm, automatically identifying artefacts cycles to be removed to clean the time series data. This can be seen around the boxes indicated 2610, 2620, 2630, 2640, 2650 between the predetermined period of the screen 2600 where a piezo sensor 2602, ECG leads 2604 are at least used.

As shown in FIG. 25G, the automated machine learning engine may use algorithms that match a template 2700, which may be optimised, to find all valid respiratory cycles 2701 that exist in the recorded time series data 2702 in relation to the respiratory cycle through obtained signals from piezo sensor. In a preferred embodiment, the automated machine learning engine may have the following list of predetermined feature thresholds such as but not limited to:
Max time width (S-E) ˜3.9 seconds; (2704)
Max amplitude/height ˜0.07 volts; (2706)
Time distance between Distinctive Peak P1 and P2 ˜0.6 seconds; (2708)
Time distance between Distinctive Peak P2 and P3 ˜0.6 seconds; (2710)
Standard Deviation (a) of Cardiac Conduction Time˜±1.66; (2712)
Standard Deviation (a) of Refill Time˜±28.3; (2714)
Standard Deviation (a) of Ejection Time˜±7.68; (2716)
Standard Deviation (a) of Aortic Volume Shift˜±47.35; (2718)
Heart Rate Variability (HRV) in the segment S-E˜684 ms; (2720)
Root Mean Square of Successive Differences (RMSSD) within the segment S-E ˜40 ms; (2722) where

$$RMSSD = \sqrt{\frac{1}{N - 1} {\sum }_{i = 1}^{N} {( {RR}_{i + 1} - {RR}_{i} )}^{2}}$$

For standard deviations, the reference equation is

$$σ = \sqrt{\frac{\sum {( {x}_{i} - μ )}^{2}}{N}}$$

where,
σ=population standard deviation;
N=the size of the population;
xᵢ=each value from the population;
μ=the population mean.

As shown in FIG. 25H, the decision flow diagram 2800 of the artefact elimination methodology is illustrated. The matching analysis based on machine learning involves an advanced motif template matching, using angular positions of multi-feature points, within the time period defined by a respiratory cycle. This automatic visual matching approach mimic a cognitive interactive method to match and eliminate artefacts. The similarity score is a predictive outcome of a trained machine learning algorithm, automatically identifying artefacts cycles to be removed to clean the time series data. The training input data 2802 is communicated to a machine learning engine 2804 that is a supervised Machine Learning (SML) methodology as an artefact predictor Model. The Training input data 2802 may have a series of peaks in which the processor may be able to detect significant relevant peaks 2808, in which it is processed for identifying the peak and time-based respiratory cycle (RC) 2810. The processor then use any of the described list of predetermined feature thresholds relating to multi-feature calculations for each of the RCs 2812 to normalize feature space representing RCs 2814, in which the processor then conducts a motif sequence search 2816 using the RC template 2818 to identify RCs with high similarity score 2820, in which the threshold for similarity 2822 is set to be equal or greater than 75%. When the similarity is equal or greater than 75%, the system classifies it as a valid respiratory cycle 2824; and when the similarity is less than 75%, the system classifies it as an invalid respiratory cycle or an artefact 2826. The system can then annotate 2830 and be part of training targets 2828 that are communicated to the machine learning engine 2804, in which the prediction based artefact detection and elimination 2806 optimises and refines the time series data.

As shown in FIGS. 19 and 20, which displays the objective of the overall system for early heart failure detection, physiological parameters related to heart failure collected from the sensor assemblies 1002 can be used to calculate Early Warning Scores for patients that are at risk of heart failure. The goal and advantage of the early detection (prior to heart failure systems) leading to early intervention, with a calculation of likelihood of the patient having heart failure at a specific point in time. The Early Warning Score based on artificial intelligence (EWS AI) also include phenotyping data from the individual including family history, medical history and other symptoms. The goal of the EWS AI is even earlier detection for even earlier intervention and better patient outcomes resulting from the use of this system. A patient who may have been identified with heart failure may be monitored 2200. The patient may position the sensor assembly/assemblies 1002 to any of the predetermined locations for obtaining the hemodynamic profile of the patient. The physiological signals sensed by the sensor assemblies 1002 may be transmitted to a processor 1100 in which the digital processing unit may display the data on the smartphone or a notification system 1700. The illegible text and graphs are merely just representative of type of information that may be displayed on smartphone. The analysed data may then be sent to and from the cloud 2004 or server, or to an artificial intelligence program or application 2000, which may be stored and analysed. The analysed data may then be analysed by the program 2000 or displayed on a clinician portal 1800. For example, the analysed data may be presented in a form of a graph 2300 as shown in FIG. 20 for a clinician to read the analysed likelihood of heart failure percentage 2302 over a pre-symptomatic time frame or interval 2304. The graph 2302 may have at least one parameter of the following group of: physiological 2306, physiological and phenotyping 2308, physiological and phenotyping and AI 2310. The diagnostic region or interval 2312 may stand out for easy viewing by the patient and/or clinician, in which the resulting analysis of the percentage of likelihood of heart failure 2302 may be notified to the patient. It may be appreciated that this is not the only graph displayed and is not limited to other graphs being displayed that provides meaningful information for the clinician to actively consult and manage the patient's heart health. In view of the analysed data, the processor 1100 may create a care plan which may be reviewed by a clinician who can update or optimise the care plan 1600 based on the analysed data 1850 or results and/or consultation in which other parameters about the patient may be noted or entered into the program 1002 or an artificial intelligence application 2000 for further processing. The consult may be in person or by distance, such as a telehealth consult 1852/1584. Once the care plan 1600 is created and relayed to the patient, the patient undertakes care plan 1600 and the effect of care plan may be monitored over a predetermined time 1860. In which historical analysed data regarding to cardiac function and condition may be compared with current measurements.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

1-28. (canceled)

29. A device for hemodynamic monitoring, the device comprising:
a processor having a digital signal processing unit, wherein the digital signal processing unit is configured to receive and process physiological signals from at least one sensor assembly having two or more sensors; wherein the processor is configured to trigger synchronized signals to the two or more sensors of the first sensor assembly for making measurement at the same time; wherein at the two or more sensors comprises a piezoelectric sensor and a piezoresistive sensor; and
the processor comprises a program having executable instructions that when executed on the processor, the processor is configured to execute the steps of:
synchronizing processed signals for each sensor assembly by calculating a phase shift from the piezoelectric sensor relative to the piezoresistive sensor;
mapping the synchronized signals as waveforms for each sensor;
conducting a cardiac cycle template matching for identifying signal parts as waveforms related to identified cardiac cycles;
annotating morphological features;
predetermining waveform time intervals to calculate at least one cardiac function parameter as a data value;
normalizing the calculated time intervals to a fixed cardiac cycle length;
calculating an average for each of the normalized predetermined time intervals; and
performing a step of differential analysis between the calculated data value and a set of referencing cardiac health parameters to determine a condition of the heart.

30. The device according to claim 29, wherein the sensor assembly comprises a force sensitive resistor, a displacement sensor, and an electrocardiogram electrode; wherein the electrocardiogram electrode is embedded in the sensor assembly.

31. The device according to claim 29, further comprising non-transition memory configured to allow the processor to: storing a condition data value corresponding to a first use of the at least one sensor assembly at a predetermined position.

32. The device according to claim 31, wherein the differential analysis step comprising the step of comparing a second condition data value corresponding to a second use of the least one sensor assembly at the predetermined position, and determining a progression of heart condition based on a difference in condition data values from the first use and the second use.

33. The device according to claim 32, wherein the processor is further configured to: providing an alert to the subject, when the determined progression is worse compared to a previous use.

34. The device according to claim 29, wherein the differential analysis step comprising the step of determining heart rate variability, pre ejection periods, isovolumetric contraction times, ejection times, and cardiac conduction times based on physiological signals received from a first sensor assembly, when the sensor assembly is positioned over or in the vicinity of a heart of the subject; and wherein differential analysis step comprises the step of determining events from a cardiac cycle based on received physiological signals, wherein the determined events correspond to at least one heart function selected from the group of: closure of the semilunar valves, ventricular blood refill period, cardiac conduction time, isovolumetric cardiac contraction time, ejection period from the cardiac output valves opening, cardiac output valves closing, and cardiac output.

35. The device according to claim 34, further comprising a second sensor assembly comprising a second group of sensors, wherein the second sensor assembly is positioned at a different predetermined location to the first sensor assembly.

36. The device according to claim 35, wherein the first sensor assembly is positioned over the heart, and the second sensor assembly is positioned on the arm, the processor is configured to derive at least one of the group selected from: the duration of first and/or second cardiac sounds, and the duration of the cardiac phases; and wherein the step of the differential analysis comprises the step of deriving the pulse transit time; wherein the differential analysis step further comprises the step of deriving central blood pressure and vessel stiffness based on relative timing between aortic valves opening and closing and waveform shapes of the received physiological signals.

37. The device according to claim 35, wherein the calculating step comprises the step of calculating of average for each of predetermined waveform amplitudes, wherein the differential analysis step further comprises the step of deriving the difference in amplitudes of the force sensors at predetermined time intervals in the force signals to determine data values for: A) calibrated peak amplitude for expansion, and B) calibrated peak amplitude for contraction; and wherein the different analysis step comprises the step of deriving elasticity of the vessel based on a ratio of A:B; and wherein the differential analysis step further comprises the step of determining a timing difference of the received signals from displacement sensors relative to the electrocardiogram electrode to determine data values for: C) time at which vessel expands, and D) time at which vessel contracts; and wherein the differential analysis step further comprises the step of generating an indication of the central hemodynamic function of the subject from a ratio of (A/C):(B/D) obtained from different predetermined locations of the sensor assemblies.

38. The device according to claim 35, wherein the first sensor assembly is positioned over the cardiac apex, and the second sensor assembly is positioned over the suprasternal notch or over an aortic arch such that the processor is adapted to determine at least one heart function through the differential analysis step.

39. The device according to claim 35, wherein the first sensor assembly is positioned over the cardiac apex of the subject, and the second sensor assembly is positioned at the aortic auscultation position of the subject, such that the processor is adapted to determine the at least one heart function relating to the left chambers of the heart through the differential analysis step.

40. The device according to claim 35, wherein the first sensor assembly is positioned over the cardiac apex of the subject, and the second sensor assembly is positioned at the pulmonary valve auscultation position of the subject such that the processor is configured to determine the at least one heart function relating to the right chambers of the heart through the differential analysis step.

41. The device according to claim 35, wherein the first sensor assembly and the second sensor assembly are positioned at adjacent locations over the top of the heart such that the processor is adapted to derive pulse elasticity and pulse timing, which then the processor determines arteria stiffness and blood pressure through the differential analysis step.

42. The device according to claim 29, wherein the differential analysis step comprising the step of determining a status of a jugular venous pulse, based on physiological signals received from a first sensor assembly which correlates to various pressure changes in the right atrium, when the sensor assembly is positioned over or in the vicinity of the jugular vein in the neck of the subject.

43. The device according to claim 29, wherein the processor is configured to execute the step of calculating measurement of variance after the calculation of average for the normalized predetermined time intervals.

44. A device for hemodynamic monitoring, the device comprising:
a processor having a digital signal processing unit, wherein the digital signal processing unit is configured to receive and process physiological signals from at least one sensor assembly having two or more sensors; wherein the processor is configured to trigger synchronized signals to the two or more sensors of the first sensor assembly for making measurement at the same time; wherein at the two or more sensors comprises a piezoelectric sensor and a piezoresistive sensor; and
the processor comprises a program having executable instructions that when executed on the processor, the processor is configured to execute the steps of:
synchronizing processed signals for each sensor assembly by calculating a phase shift from the piezoelectric sensor relative to the piezoresistive sensor;
mapping the synchronized signals as waveforms for each sensor;
conducting a cardiac cycle template matching for identifying signal parts as waveforms related to identified cardiac cycles;
annotating morphological features;
predetermining waveform amplitudes to calculate at least one cardiac function parameter as a data value;
calculating an average for each of the predetermined waveform amplitudes; and
performing a step of differential analysis between the calculated data value and a set of referencing cardiac health parameters to determine a condition of the heart.

45. The device according to claim 44, wherein the processor is configured to trigger synchronized signals to the first sensor assembly and the second sensor assembly at two different locations for making measurement at the same time; and wherein the differential analysis step further comprises the step of determining an ejection fraction of the heart based on subtracting physiological signals from the displacement sensor in the first sensor assembly and the second sensor assembly.

46. The device according to claim 44, wherein the processor is configured to execute the step of calculating measurement of variance after the calculation of average for the predetermined waveform amplitudes.

47. The device according to claim 46, wherein the annotation of morphological features comprises potential artefact signals.

48. The device according to claim 47, wherein the processor can be configured to normalize synchronized processed signals between the variance calculation step and the differential analysis step, wherein for normalization, the processor is configured to execute the steps of:
removing the identified potential artefact signals from the synchronized waveforms; joining identified signal parts to the synchronized waveforms with removed artefact signals to form a cleaned signal as waveform; and
recalculating the new average and measurement of variances for each of the waveform amplitudes and the time intervals from the cleaned waveform, wherein the recalculated time intervals, for the at least one cardiac function parameter, are normalized with respect to length of cardiac cycle.
